(19) United States
(12) Patent Application Publication
(10) Pub. No.: US 2023/0355537 A1
(43) Pub. Date: Nov. 9, 2023

(54) PROMOTING TRAINED IMMUNITY WITH THERAPEUTIC NANOBIOLOGIC COMPOSITIONS

(71) Applicants: Icahn School of Medicine at Mount Sinai, New York, NY (US); STICHTING KATHOLIEKE UNIVERSITEIT, Nijmegen (NL)

(72) Inventors: Willem Mulder, Eindhoven (NL); Jordi Ochando, New York, NY (US); Zahi Fayad, Larchmont, NY (US); Mihai Netea, Numegen (NL); Leo Joosten, Numegen (NL)

(21) Appl. No.: 18/076,759

(22) Filed: Dec. 7, 2022

Related U.S. Application Data
(63) Continuation of application No. 17/743,342, filed on May 12, 2022, now abandoned.
Application No. 17/743,342 is a continuation of application No. 16/862,570, filed on Apr. 30, 2020, now abandoned.
Application No. 16/862,570 is a continuation of application No. PCT/US2018/061935, filed on Nov. 20, 2018.
(60) Provisional application No. 62/589,054, filed on Nov. 21, 2017.

(51) Int. Cl. A61K 47/64; A61K 39/39; C07K 14/775; A61K 47/54; A61K 9/51

(52) U.S. Cl. CPC A61K 9/5123; A61K 39/39; A61K 47/544; A61K 47/64; C07K 14/775; A61K 38/00

(57) ABSTRACT

The invention relates to therapeutic nanobiologic compositions and methods of treating patients who have cancer, by promoting trained immunity, which is the long-term increased responsiveness, the result of metabolic and epigenetic re-wiring of myeloid cells and their stem cells and progenitors in the bone marrow and spleen and blood induced by a primary insult, and characterized by increased cytokine excretion after re- stimulation with one or multiple secondary stimuli.

### CROSS-REFERENCE TO RELATED APPLICATIONS

This application is a continuation and claims priority benefit under 35 USC 366(c) to international application PCT/US18/61986 filed Nov. 20, 2018, which claims priority benefit to U.S. Pat. Application 62589054 filed Nov. 21, 2017, both of which are hereby incorporated by reference in their entirety.

### STATEMENT REGARDING FEDERALLY SPONSORED R&D

This invention was made with government support under grant R01 HL1 18440 awarded by the National institutes of Health. The government has certain rights in the invention.

### FIELD OF THE INVENTION

The invention relates to therapeutic nanobiologic compositions and methods of treating patients who have cancer or infections, by promoting trained immunity, which is a secondary long-term hyper-responsiveness, as manifested by increased cytokine excretion caused by metabolic and epigenetic rewiring, by using a nanobiologic composition for stimulation of myeloid cells and their progenitors and stem cells in the bone marrow, spleen and blood.

### BACKGROUND OF THE INVENTION

### Description of the Related Art

Current treatments for patients who suffer from cancer can be inadequate. Patients who have cancer, are in need of a treatment paradigm that is durable, and that does not cause more problems in side effects than the primary treatment itsell.

Currert cancer therapy may involve surgery, chemotherapy, hormonal therapy and/or radiation treatment to eradicate neoplastic cells in a patient (see, for example, Stockdale, 1998, Medicine, vol. 3. Rubenstein and Federman, eds.. Chapter 12, Section IV). Recently, cancer therapy can also involve biological therapy or immunotherapy. All of these approaches pose significant drawbacks for the patient. Surgery, for example, may be contrandicated due to the health of a patient or may be unacceptable to the patient.

Additionally, surgery may not completely remove neoplastic tissue. Radiation therapy is only effective when the neoplastic tissue exhibits a higher sensitivity to radiation than normal tissue. Radiation therapy can also often elicit serious side effects. Hormonal therapy is rarely given as a single agent. Although hormonal therapy can be effective, it is often used to prevent or delay recurrence of cancer after other treatments have removed the majority of cancer cells. Biological therapies and immunotherapies are limited in number and may produce side effects such as rashes or swellings, flu-like symptoms, including fever, chills and fatigue, digestive tract problems or allergic reactions.

With respect to chemotherapy, there are a variety of chemotherapeutic agents available for treatment of cancer. A majority of cancer chemotherapeutics act by inhibiting DNA synthesis, either directly, or indirectly by inhibiting the biosynthesis of deoxyribonucleotide triphosphate precursors, to prevent DNA replication and concomitant cell division. Gilman et al., Goodman and Gilman’s: The Pharmacological Basis of Therapeutics, Tenth Ed. (McGraw Hill, New York). Despite availability of a variety of chemotherapeutic agents, chemotherapy has many drawbacks. Stockdale, Medicine, vol. 3, Rubenstein and Federman, eds., ch. 12, sect. 10, 1998. Almost all chemotherapeutic agents are toxic, and chemotherapy causes significant, and often dangerous side effects including severe nausea, bone marrow depression, and immunosuppression. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even if those agents act by different mechanism from those of the drugs used in the specific treatment. This phenomenon is referred to as pleiotropic drug or multidrug resistance. Because of the drug resistance, many cancers prove refractory to standard chemotherapeutic treatment protocols. Still, there is a significant need for safe and effective methods of treating, preventing and managing cancer, and other diseases and conditions caused by defective trained immunity, particularly for diseases that are refractory to standard treatments, such as surgery, radiation therapy, chemotherapy and hormonal therapy, while reducing or avoiding the toxicities and/or side effects associated with the conventional therapies.

In recent decades, our knowledge of the immune system has yielded several promising immunotherapeutic approaches that provide great benefits to patients. Today’s clinically relevant immunotherapies engage either effector molecules, such as cytokines, or the cellular stage of adaptive immunity. In autoimmune and autoinflammatory diseases, anti-cytokine therapies can successfully neutralize bioactive cytokines, while the most intensely used immunotherapy in cancer patients comprises the application of checkpoint-inhibitor drugs. These drugs take the brake off T-cells, enabling them to eliminate tumor cells. Antibodies specific to cytotoxic T lymphocyte-associated antigen 4 (CTLA-4), as well as antibodies against programmed cell death protein 1 (PD1) and its ligand PD-L1, are the most advanced in terms of clinical application. Allematively, adoptive T-cell therapies involve collecting these cells from a patient, expanding their number in culture, and reintroducing them into the body. In culture, T-cells can also be genetically modified to increase their affinity for tumor cells. Dendritic cell therapy is another therapeutic modality that has gained a lot of traction. It involves presenting tumor- specific antigens to dendritic cells, either ex vivo or in vivo, to induce a tumor-specific T-cell response.

Whereas the aforementioned immunotherapeutic approaches focus on T lymphocytes, which are cells from the adaptive immune system, there is still a need for improved therapies.

### SUMMARY OF THE INVENTION

Accordingly, to address these and other deficiencies in the prior art, in a preferred embodiment of the invention, the invention provides nanobiologics that engage the innate immune system, in particular myeloid cells and their stem cells and progenitors in the bone marrow, blood and spleen, and methods of treating a patient in need thereof with a therapeutic agent for promoting trained immunity.

Trained Immunity is defined by a secondary long-term hyper-responsiveness, as manifested by increased cytokine excretion caused by metabolic and epigenetic rewiring, to re stimulation after a primary insult of myeloid cells and their progenitors and stem cells in the bone marrow, spleen and blood. Trained Immunity (also called innate immune memory) is also defined by a long-term increased responsiveness (e.g. high cytokine production) after re stimulation with a secondary stimulus of myeloid innate immune cells, being induced by a primary insult stimulating these cells or their progenitors and stem cells in the bone marrow and spleen, and mediated by epigenetic, metabolic and transcriptional rewiring.

### Treating Cancer or Sepsis

In a non-limiting preferred embodiment of the Invention, there is provided a method of treating a patient by inducing trained immunity to treat cancer or sepsis:
(i) administering to said patient a nanobiologic composition in an amount effective to promote a hyper-responsive innate immune response,
   wherein the nanobiologic composition comprises (i) a nanoscale assembly, having (ii) an innate immune response promoter drug incorporated in the nanoscale assembly,
   wherein the nanoscale assembly is a multi-component carrier composition comprising: (a) phospholipids.
   (b) apolipoprotein A-I (apoA-I) or a peptide mimetic of apoA-I,
   wherein said nanobiologic, in an aqueous environment, is a nanodisc or nanosphere with size between about 8 nm and 400 nm in diameter,
   wherein the nanobiologic is functionalized with a molecular structure that activates or binds to the pathogen recognizing receptors Dectin-I or NOD2 to induce trained immunity in myeloid cells and their stem cells and progenitors in the bone marrow, blood and spleen, wherein the molecular structures that activate or bind to Dectin-I include, but are not limited to, b-glucans and its derivatives such as 11-13 gluco-oligomers, wherein the molecular structures that activate or bind to NOD2 include, but are not limited to, peptidoglycans and its derivatives such as muramyl dipeptide (MDP) and muramyl tripeptide (MTP),
   wherein the nanoscale assembly delivers the trained immunity-promoter molecular structures to myeloid cells, myeloid progenitor cells or hematopoietic stem cells in bone marrow, blood and/or spleen of the patient.
whereby in the patient a hyper-responsive innate immune response caused by trained immunity is promoted, and cancer or sepsis is treated.

In a non-limiting preferred embodiment of the invention, the nanoscale assembly also includes (c) a hydrophobic matrix comprising one or more triglycerides, fatty acid esters, hydrophobic polymers, or sterol esters, or a combination thereof.

In another non-limiting preferred embodiment of the invention, the nanoscale assembly also includes (c) a hydrophobic matrix comprising one or more triglycerides, fatty acid esters, hydrophobic polymers, or sterol esters, or a combination thereof, and (d) cholesterol.

### Improving the Efficacy of Checkpoint Inhibitors

In another non-limiting preferred embodiment of the invention, the invention comprises a method of treating a patient by improving the efficacy of a checkpoint inhibitor treatment by inducing trained immunity:
(1) administering to said patient a nanobiologic composition in an amount effective to promote a hyper-responsive innate immune response,
   wherein the nanobiologic composition comprises (i) a nanoscale assembly, having (ii) an innate immune response promoter drug incorporated in the nanoscale assembly,
   wherein the nanoscale assembly is a multi-component carrier composition comprising: (a) phospholipids, and,
   (b) apolipoprotein A-I (apoA-I) or a peptide mimetic of apoA-I,
   wherein said nanobiologic, in an aqueous environment, is a nanodisc or nanosphere with size between about 8 nm and 400 nm in diameter,
   wherein the nanobiologic is functionalized with a molecular structure that activates or binds to the pathogen recognizing receptors Dectin-I or NOD2 to induce trained immunity in myeloid cells and their stem cells and progenitors in the bone marrow, blood and spleen, wherein the molecular structures that activate or bind to Dectin-I include, but are not limited to, Beta-glucans and its derivatives such as 11-13 gluco-oligomers, wherein the molecular structures that activate or bind to NOD2 include, but are not limited to, peptidoglycans and its derivatives such as muramyl dipeptide (MDP) and muramyl tripeptide (MTP),
   wherein the nanoscale assembly delivers the trained immunity-promoter molecular structures to myeloid cells, myeloid progenitor cells or hematopoietic stem cells in bone marrow, blood and/or spleen of the patient;
   whereby in the patient a hyper-responsive innate immune response caused by trained immunity is promoted; and
(2) administering to said patient a checkpoint inhibitor;
   whereby promoting the hyper-responsive innate immune response caused by trained immunity improves the efficacy of checkpoint inhibitor therapy.

In a non-limiting preferred embodiment of the invention, the nanoscale assembly also includes (c) a hydrophobic matrix comprising one or more triglycerides, fatty acid esters, hydrophobic polymers, or sterol esters, or a combination thereof.

In another non-limiting preferred embodiment of the invention, the nanoscale assembly also includes (c) a hydrophobic matrix comprising one or more triglycerides, fatty acid esters, hydrophobic polymers, or sterol esters, or a combination thereof, and (d) cholesterol.

### Promoting Long Term Tumor Remission

In a non-limiting preferred embodiment of the invention, there is provided a method of promoting long-term tumor remission in a patient that has received a cancer diagnosis, comprising the following steps:
(1) administering to said patient a standard regimen of treatment specific for the cancer of the patient, including chemotherapy, radiation therapy, immunotherapy, and therapeutically effective combinations thereof
(2) administering to said patient a nanobiologic composition in an amount effective to promote a long-term hyper-responsive innate immune response,
   wherein the nanobiologic composition comprises (i) a nanoscale assembly, having (ii) an innate immune response promoter drug incorporated in the nanoscale assembly, wherein the nanoscale assembly is a multi-component carrier composition comprising: (a) phospholipids, and,
   (b) apolipoprotein A-I (apoA-I) or a peptide mimetic of apoA-I,
   wherein the promoter drug is a molecular structure that activates or binds to the pathogen recognizing receptors Dectin-I or NOD2, wherein the molecular structures that activate or bind to Dectin-I include, but are not limited to, Beta-glucans and its derivatives such as 11-13 gluco-oligomers, wherein the molecular structures that activate or bind to NOD2 include, but are not limited to, peptidoglycans and its derivatives such as muramyl dipeptide (MDP) and muramyl tripeptide (MTP),
   wherein said nanobiologic, in an aqueous environment, is a nanodisc or nanosphere with size between about 8 nm and 400 nm in diameter,
   wherein the nanoscale assembly delivers the promoter drug to myeloid cells, myeloid progenitor cells or hematopoietic stem cells in bone marrow, blood and/or spleen of the patient,
   whereby in the patient a hyper-responsive innate immune response caused by trained immunity is promoted; and optionally,
(3) administering to said patient a checkpoint inhibitor;
   whereby promoting the hyper-responsive innate immune response caused by trained immunity improves the efficacy of checkpoint inhibitor therapy.

In a non-limiting preferred embodiment of the invention, the nanoscale assembly also includes (c) a hydrophobic matrix comprising one or more triglycerides, fatty acid esters, hydrophobic polymers, or sterol esters, or a combination thereof.

In another non-limiting preferred embodiment of the invention, the nanoscale assembly also includes (c) a hydrophobic matrix comprising one or more triglycerides, fatty acid esters, hydrophobic polymers, or sterol esters, or a combination thereof, and (d) cholesterol.

### Providing Long-Term Innate Trained Immunity

In a non-limiting preferred embodiment of the invention, there is provided a method of treating a patient affected by detective trained immunity (immunoparalysis) to promote in said patient a long-term hyper-responsive innate immune response, comprising:
(1) administering to said patient a nanobiologic composition in an amount effective to promote a hyper-responsive innate Immune response,
   wherein the nanobiologic composition comprises (i) a nanoscale assembly, having (ii) a promoter drug incorporated in the nanoscale assembly,
   wherein the nanoscale assembly is a multi-component carrier composition comprising: (a) phospholipids, and,
   (b) apoA-I or a peptide mimetic of apoA-I,
   wherein the promoter drug is a molecular structure that activates or binds to the pathogen recognizing receptors Declin-I or NOD2 to induce trained immunity in myeloid cells and their stem cells and progenitors in the bone marrow, blood and spleen, wherein the molecular structures that activate or bind to Dectin-I include, but are not limited to, Beta-glucans and its denvatives such as 11-13 gluco-oligomers, wherein the molecular structures that activate or bind to NOD2 include, but are not limited to, peptidoglycans and Its derivatives such as muramyl dipeptide (MDP) and muramyl tripeptide (MTP),
   wherein said nanobiologic, in an aqueous environment, self-assembles into a nanodisc or nanosphere with size between about 8 nm and 400 nm in diameter,
   wherein the nanoscale assembly delivers the drug to myeloid cells, myeloid progenitor cells or hematopoietic stem cells in bone marrow, blood and/or spleen of the patient,
   and whereby in the patient the hyper-responsive innate immune response is promoted; and optionally.
(2) administering to said patient a checkpoint inhibitor after administering the nanobiologic composition,
   whereby promoting the hyper-responsive innate immune response caused by trained immunity improves the efficacy of checkpoint inhibitor therapy.

In a non-limiting preferred embodiment of the invention, the nanoscale assembly also includes (c) a hydrophobic matrix comprising one or more triglycerides, fatty acid esters, hydrophobic polymers, or sterol esters, or a combination thereof.

In another non-limiting preferred embodiment of the invention, the nanoscale assembly also includes (c) a hydrophobic matrix comprising one or more triglycerides, fatty acid esters, hydrophobic polymers, or sterol esters, or a combination thereof, and (d) cholesterol.

### Pet Imaging Accumulation of Drug Within the Body

In a non-limiting preferred embodiment of the invention, there is provided a nanobiologic composition for imaging accumulation of a nanobiologic within bone marrow, blood, and/or spleen, of a patient affected by trained immunity, comprising: (i) a nanoscale assembly, having (ii) an promoter drug incorporated in the nanoscale assembly, and (ili) a position emission tomography (PET) radioisotope incorporated in the nanoscale assembly, wherein the nanoscale assembly is a multi-component carrier composition comprising: (a) phospholipids, and, (b) apoA-1 or a peptide mimetic of apoA-1,
wherein the promoter drug is a molecular structure that activates or binds to the pathogen recognizing receptors Declin-1 or NOD2 to induce trained immunity in myeloid cells and their stem cells and progenitors in the bone marrow, blood and spleen, wherein the molecular structures that activate or bind to Dedin-I include, but are not limited to, Beta-glucans and its derivatives such as 11-13 gluco-oligomers, wherein the molecular structures that activate or bind to NOD2 include, but are not limited to, peptidoglycans and its derivatives such as muramyl dipeptide (MDP) and muramyl tripeptide (MTP),
wherein the PET imaging radioisotope is selected from 89Zr, 1241, 64Cu, 18F and 86Y, and wherein the PET imaging radioisotope is complexed to the nanobiologic using a suitable chelating agent to form a stable nanobiologic-radioisotope chelate,
wherein said nanobiologic, in an aqueous environment, self-assembles into a nanodisc or nanosphere with size between about 8 nm and 400 nm in diameter,
wherein the nanoscale assembly delivers the stable nanobiologic-radioisotope chelate to myeloid cells, myeloid progenitor cells or hematopoietic stem cells in bone marrow, blood and/or spleen of the patient.

In a non-limiting preferred embodiment of the invention, the nanoscale assembly also includes (c) a hydrophobic matrix comprising one or more triglycerides, fatty acid esters, hydrophobic polymers, or sterol esters, or a combination thereof.

In another non-limiting preferred embodiment of the invention, the nanoscale assembly also includes (c) a hydrophobic matrix comprising one or more triglycerides, fatty acid esters, hydrophobic polymers, or sterol esters, or a combination thereof, and (d) cholesterol.

In a non-limiting preferred embodiment of the invention, there is provided a method of positron emission tomography (PET) imaging the accumulation of a nanobiologic within bone marrow, blood, and/or spleen, of a patient affected by trained immunity, comprising:
(1) administering to said patient a nanobiologic composition in an amount effective to promote a hyper-responsive innate Immune response,
   wherein the nanobiologic composition comprises (i) a nanoscale assembly, having (ii) an promoter drug incorporated in the nanoscale assembly, and (iii) a positron emission tomography (PET) radioisolope incorporated in the nanoscale assembly,
   wherein the nanoscale assembly is a multi-component carrier composition comprising: (a) phospholipids, and,
   (b) apoA-1 or a peptide mimetic of apoA-1,
   wherein the promoter drug is a molecular structure that activates or binds to the pathogen recognizing receptors Declin-1 or NOD2 to induce trained immunity in myeloid cells and their stem cells and progenitors in the bone marrow, blood and spleen, wherein the molecular structures that activate or bind to Dedin-I include, but are not limited to, Beta-glucans and its derivatives such as 11-13 gluco-oligomers, wherein the molecular structures that activate or bind to NOD2 include, but are not limited to, peptidoglycans and its derivatives such as muramyl dipeptide (MDP) and muramyl tripeptide (MTP),
   wherein the PET imaging radioisotope is selected from 89Zr, 1241, 84Cu, 18F and 86Y, and
   wherein the PET imaging radioisotope is complexed to the nanobiologic using a suitable chelating agent to form a stable nanobiologic-radioisotope chelate,
   wherein said nanobiologic, in an aqueous environment, self-assembles into a nanodisc or nanosphere with size between about 8 nm and 400 nm in diameter,
   wherein the nanoscale assembly delivers the stable nanobiologic-radioisotope chelate to myeloid cells, myeloid progenitor cells or hematopoietic stem cells in bone marrow, blood and/or spleen of the patient, and
(2) performing PET imaging of the patient to visualize biodistribution of the stable nanobiologic-radioisotope chelate within the bone marrow, blood, and/or spleen of the patient’s body.

In a non-limiting preferred embodiment of the invention, the nanoscale assembly also includes (c) a hydrophobic matrix comprising one or more triglycerides, fatty acid esters, hydrophobic polymers, or sterol esters, or a combination thereof.

In another non-limiting preferred embodiment of the invention, the nanoscale assembly also includes (c) a hydrophobic matrix comprising one or more triglycerides, fatty acid esters, hydrophobic polymers, or sterol esters, or a combination thereof, and (d) cholesterol.

In a non-limiting preferred embodiment, the method of radiopharmaceutical imaging comprises an additional step of administering to said patient a checkpoint inhibitor with the nanobiologic composition,

whereby promoting the hyper-responsive innate immune response caused by trained immunity improves the efficacy of checkpoint inhibitor therapy.

In a non-limiting preferred embodiment of the invention, there is provided a method wherein the hyper-responsive innate immune response is promoted for at least 7 to 30 days.

In a non-limiting preferred embodiment of the invention, there is provided a method wherein the hyper-responsive innate immune response is promoted for at least 30 to 100 days.

In a non-limiting preferred embodiment of the invention, there is provided a method wherein the hyper-responsive innate immune response is promoted for more than 100 days and up to 3 years.

In a non-limiting preferred embodiment of the invention, there is provided a method wherein the patient affected by trained immunity suffers from cancer of the bladder, blood vessels, bone, brain, breast, cervix, chest, colon, endrometrium, esophagus, eye, head, kidney, liver, lymph nodes, lung, mouth, neck, ovaries, pancreas, prostate, rectum, skin, stomach, testis, throat, thyroid, urothelium, or uterus.

In a non-limiting preferred embodiment of the invention, there is provided a method wherein the nanobiologic composition is administered once and wherein the hyper-responsive innate immune response is promoted for at least 30 days.

In a non-limiting preferred embodiment of the invention, there is provided a method wherein the nanobiologic composition is administered at least once per day in each day of a multiple dosing regimen, and wherein the hyper-responsive innate immune response is promoted for at least 30 days.

In a non-limiting preferred embodiment of the invention, the promoter drug is MDP, MTP, b-glucan, polymers of sugars, ox-LDL, BCG, bacterial peptidoglycans, viral peptides, a drug or compound or polymer that activates or binds to Dectin-i or NOD2, a promoter of the inflammasome, a promoter of metabolic pathways, and/or a promoter of epigenetic pathways within a hematopoietic stem cell (HSC), a common myeloid progenitor (CMP), or a myeloid cell.

In a non-limiting preferred embodiment of the invention, there is provided a method wherein trained Immunity is defined by a secondary hyper-responsiveness, as manifested by increased cytokine excretion caused by metabolic and epigenetic rewiring, to re-stimulation after administration of the nanobiologic to generate a primary insult of myeloid cells and their progenitors and stem cells in the bone marrow.

In a non-limiting preferred embodiment of the invention, there is provided a method wherein trained immunity is defined by a long-term increased responsiveness from high cytokine production after administration of the nanobiologic to generate a secondary stimulus of myeloid innate immune cells, being induced after administration of the nanobiologic to generate a primary insult stimulating these cells or their progenitors and stem cells in the bone marrow, and mediated by epigenetic, metabolic and transcriptional rewiring.

In a non-limiting preferred embodiment of the invention, there is provided a method wherein the promoter drug is a NOD2 receptor promoter, an mTOR promoter, a ribosomal protein S6 kinase beta-I (S6K1) promoter, a histone H3K27 demethylase promoter, a BET bromodomain blockade promoter, a promoter of histone methyltransferases and acethyltransferases, a promoter of DNA methyltransferases and acethyltransferases, an inflammasome promoter, a Serine/threonine kinase Akt promoter, an Promoter of Hypoxia-inducible factor 1-alpha, also known as HIF-I-a, inhibitors of histone and DNA demethylases and deacetylases, and a mixture of one or more thereof.

In a non-limiting preferred embodiment of the invention, there is provided a method wherein the patient has severe sepsis or is in septic shock.

In a non-limiting preferred embodiment of the invention, there is provided a method wherein the patient has sepsis associated with a bacterial, viral or fungal infection of the lungs, abdomen, kidney, or bloodstream.

In a non-limiting preferred embodiment of the invention, there is provided a method wherein the nanobiologic composition is administered in a treatment regimen comprising two or more doses to the patient to generate an accumulation of drug in myeloid cells, myeloid progenitor cells, and hematopoietic stem cells in the bone marrow, blood and/or spleen.

In a non-limiting preferred embodiment of the invention, there is provided a method comprising co-administering a cancer drug as a combination therapy with the nanobiologic composition.

### Nanobiologic Composition

In a non-limiting preferred embodiment of the invention, there is provided a nanobiologic composition for promoting trained immunity, comprising:
(i) a nanoscale assembly, having (ii) a promoter drug incorporated in the nanoscale assembly, wherein the (i) nanoscale assembly is a multi-component carrier composition comprising:
   (a) phospholipids,
   (b) apoA-1 or a peptide mimetic of apoA-I,
      wherein the promoter drug is a molecular structure that activates or binds to the pathogen recognizing receptors Dectin-I or NOD2 to induce trained immunity in myeloid cells and their stem cells and progenitors in the bone marrow, blood and spleen, wherein the molecular structures that activate or bind to Dectin-I include, but are not limited to, Beta-glucans and its derivatives such as 11-13 gluco-oligomers, wherein the molecular structures that activate or bind to NOD2 include, but are not limited to, peptidoglycans and its derivatives such as muramyl dipeptide (MDP) and muramyl tripeptide (MTP),
      wherein said nanobiologic, in an aqueous environment, self-assembles into a nanodisc or nanosphere with size between about 8 nm and 400 nm in diameter,
      wherein the nanoscale assembly delivers the drug to myeloid cells, myeloid progenitor cells or hematopoietic stem cells in bone marrow, blood and/or spleen of the patient,
      and whereby in the patient the hyper-responsive innate Immune response is promoted.

In a non-limiting preferred embodiment of the invention, the nanoscale assembly also includes (c) a hydrophobic matrix comprising one or more triglycerides, fatty acid esters, hydrophobic polymers, or sterol esters, or a combination thereof.

In another non-limiting preferred embodiment of the invention, the nanoscale assembly also includes (c) a hydrophobic matrix comprising one or more triglycerides, fatty acid esters, hydrophobic polymers, or sterol esters, or a combination thereof, and (d) cholesterol.

In a non-limiting preferred embodiment of the invention, there is provided a nanobiologic composition for promoting trained immunity wherein the promoter drug is MDP, MTP. b-glucan, polymers of sugars, ox-LDL, BCG, bacterial peptidoglycans, viral peptides, Dectin-I, a promoter of the inflammasome, a promoter of metabolic pathways, and/or a promoter of epigenetic pathways within a hematopoietic stem cell (HSC), a common myeloid progenitor (CMP), or a myeloid cell.

In a non-limiting preferred embodiment of the invention, there is provided a nanobiologic composition for promoting trained immunity wherein the promoter drug is a NOD2 receptor promoter, an mTOR promoter, a ribosomal protein S6 kinase beta-I (S6K1) promoter, an HMG-CoA reductase promoter (Statin), a histone H3K27 demethylase promoter, a BET bromodomain blockade promoter, an promoter of histone methyltransferases and acethyltransferases, an promoter of DNA methyltransferases and acethyltransferases, an inflammasome promoter, a Serine/threonine kinase Akt promoter, an Promoter of Hypoxia-inducible factor 1-alpha, also known as HIF-I-a, and a mixture of one or more thereof.

### Radiolabelled Nanobiologic

In a non-limiting preferred embodiment of the invention, there is provided a nanobiologic composition for imaging accumulation in bone marrow, blood and spleen, comprising:
(i) a nanoscale assembly, having (ii) a promoter drug incorporated in the nanoscale assembly, and (iii) a positron emission tomography (PET) imaging radioisotope incorporated in the nanoscale assembly,
   wherein the nanoscale assembly is a multi-component carrier composition comprising: (a) phospholipids, and,
(b) apoA-I or a peptide mimetic of apoA-I,
   wherein the promoter drug is a molecular structure that activates or binds to the pathogen recognizing receptors Dectin-I or NOD2 to induce trained immunity in myeloid cells and their stem cells and progenitors in the bone marrow, blood and spleen, wherein the molecular structures that activate or bind to Dectin-I include, but are not limited to, Beta-glucans and its derivatives such as 11-13 gluco-oligomers, wherein the molecular structures that activate or bind to NOD2 include, but are not limited to, peptidoglycans and its derivatives such as muramyl dipeptide (MDP) and muramyl tripeptide (MTP),
   wherein the PET imaging radioisotope is selected from 89Zr, 1241, 64Cu, 18F and 86Y, and
   wherein the PET imaging radioisotope is complexed to the nanobiologic using a suitable chelating agent to form a stable nanobiologic-radioisotope chelate,
   wherein said nanobiologic, in an aqueous environment, self-assembles into a nanodisc or nanosphere with size between about 8 nm and 400 nm in diameter,
   wherein the nanoscale assembly delivers the stable nanobiologic-radioisotope chelate to myeloid cells, myeloid progenitor cells or hematopoietic stem cells in bone marrow, blood and/or spleen of the patient.

In a non-limiting preferred embodiment of the invention, the nanoscale assembly also includes (c) a hydrophobic matrix comprising one or more triglycerides, fatty acid esters, hydrophobic polymers, or sterol esters, or a combination thereof.

In another non-limiting preferred embodiment of the invention, the nanoscale assembly also includes (c) a hydrophobic matrix comprising one or more triglycerides, fatty acid esters, hydrophobic polymers, or sterol esters, or a combination thereof, and (d) cholesterol.

### Process for Manufacturing

In a non-limiting preferred embodiment of the invention, there is provided a process for manufacturing a nanobiologic composition for inhibiting trained immunity, comprising the step,
incorporating a promoter drug into a nanoscale assembly;
   wherein the nanoscale assembly is a multi-component carrier composition comprising: (a) phospholipids, and,
   (b) apoA-I or a peptide mimetic of apoA-I,
   wherein the promoter drug is molecular structure that activates or binds to the pathogen recognizing receptors Dectin-I or NOD2 to induce trained immunity in myeloid cells and their stem cells and progenitors in the bone marrow
   wherein said nanobiologic, in an aqueous environment, self-assembles into a nanodisc or nanosphere with size between about 8 nm and 400 nm in diameter,
   wherein the nanoscale assembly delivers the drug to myeloid cells, myeloid progenitor cells or hematopoietic stem cells in bone marrow, blood and/or spleen of the patient, and whereby in the patient the hyper-responsive innate immune response is promoted.

In a non-limiting preferred embodiment of the invention, the nanoscale assembly also includes (c) a hydrophobic matrix comprising one or more triglycerides, fatty acid esters, hydrophobic polymers, or sterol esters, or a combination thereof.

In another non-limiting preferred embodiment of the invention, the nanoscale assembly also includes (c) a hydrophobic matrix comprising one or more triglycerides, fatty acid esters, hydrophobic polymers, or sterol esters, or a combination thereof, and (d) cholesterol.

In a non-limiting preferred embodiment of the invention, the nanoscale assembly also includes a phospholipid conjugated to a radioisotope chelating agent.

In a non-limiting preferred embodiment of the invention, there is provided a process for manufacturing a nanobiologic composition for inhibiting trained immunity, wherein the promoter drug is MDP, MTP, b-glucan, polymers of sugars, ox-LDL, BCG, bacterial peptidoglycans, viral peptides, Dectin-I, a promoter of the inflammasome, a promoter of metabolic pathways, and/or a promoter of epigenetic pathways within a hematopoietic stem cell (HSC), a common myeloid progenitor (CMP), or a myeloid cell.

In a non-limiting preferred embodiment of the invention, there is provided a process for manufacturing a nanobiologic composition for inhibiting trained immunity, wherein the assembly is combined using microfluidics, scale-up microfluidizer technology, sonication, organic-to-aqueous infusion, or lipid film hydration.

### BRIEF DESCRIPTION OF THE DRAWINGS

For the purpose of illustrating the invention, there are depicted in drawings certain embodiments of the invention. However, the invention is not limited to the precise arrangements and instrumentalities of the embodiments depicted in the drawings.

FIGS. 1A and 1B are graphs displaying the concentration of cytokines IL-6 (FIG. 1A) and TNF-a (FIG. 1B) of human monocytes that were exposed to a trained immunity-inducing agent (BCG, MDP or MTP-HDL) for 24 hours, after which the cells were washed and left to rest for 5 days before restimulation with LPS. The increased cytokine production shows MTP-HDL ‘s ability to induce trained immunity.

FIG. 2 shows maximum intensity projection (MIP) PET images of mice that were intravenously injected with 89Zr-labeled MTP-HDL. High uptake in the bone marrow was appreciated.

FIG. 3 is a graph of a dose-response curve obtained in C57BL/6 mice that were inoculated on their flanks with B16F10 tumor cells to grow melanoma. The animals were treated with different doses of MTP-HDL (muramyl tripeptide functionalized HDL nanobiologics) at different frequencies (1, 2. or 3 times). The tumor volume as a function of time after tumor cell inoculation and as function of different treatments is depicted.

FIG. 4 is a graph of monocytes per mL in the bone marrow showing amount over days after 3 intravenous MDP-HDL infusions vs. control.

FIG. 5 is a graph of FDG-PET imaging results of bone marrow showing control vs. MDP-HDL. The uptake of FDG, a sugar analog, is expressed as the standard uptake value (SUV).

FIG. 6 is a graph of a comparison of a PD-I inhibitor, MTP-HDL and the combination of PD-I inhibitor and MTP-HDL treatment, showing tumor volume vs. days after tumor inoculation. MTP-HDL was intravenously administered at day 8, 11 and 13 after tumor inoculation. Checkpoint inhibitor drugs were administered at day 11 and 14.

FIG. 7 is a graph of a comparison of a CTLA-4 inhibitor, MTP-HDL and the combination of CTLA-4 Inhibitor and MTP-HDL treatment, showing tumor volume vs. days after tumor inoculation. MTP-HDL was intravenously administered at day 8, 11 and 13 after tumor inoculation. Checkpoint inhibitor drugs were administered at day 11 and 14.

FIG. 8 is a graph of a comparison of a PD-I + CTLA-4 inhibitor, MTP-HDL and the combination of PD-I + CTLA-4 inhibitor and MTP-HDL treatment, showing tumor volume vs. days after tumor inoculation. MTP-HDL was intravenously administered at day 8, 11 and 13 after tumor inoculation. Checkpoint inhibitor drugs were administered at day 11 and 14. FIG. 9 is a graph of a comparison of a PD-I + CTLA-4 inhibitor, MTP-HDL and the combination of PD-I + CTLA-4 inhibitor and MTP-HDL treatment, where the treatment MTP-HDL was continued, showing tumor volume vs. days after tumor inoculation. MTP-HDL was intravenously administered at day 8, 11, 13, 15, 17 after tumor inoculation.

Checkpoint inhibitor drugs were administered at day 11, 14.

FIG. 10 is a graph of flow cytometry results at 24 hours after 3rd injection of MTP-HDL and shows percent of viable CD1 lb+ bone marrow cells vs. various treatments and PBS control.

FIG. 11 is a graph of flow cytometry results at 24 hours after 3rd injection of MTP-HDL and shows percent of viable bone marrow monocytes vs. various treatments and PBS control.

FIGS. 12A and 12B are graphs of flow cytometry results at 24 hours after 3rd injection of MTP-HDL. FIG. 12A shows percent of viable CDI lb-i- blood cells vs. various treatments and PBS control. FIG. 12B shows percent of viable CDIlb-i- spleen cells vs. various treatments and PBS control.

FIGS. 13A and 13B are graphs of flow cytometry results at 24 hours after 3rd injection of MTP-HDL FIG. 13A shows percent of viable blood monocytes vs. various treatments and PBS control. FIG. 13B shows percent of viable spleen monocytes vs. various treatments and PBS control.

FIG. 14 is an illustration of a schematic of processes that control trained immunity, at the epigenetic, cellular and systems level. The originally identified ‘trainers ‘include the fungal PAMP b-glucan and the bacterial PANP peptidoglycan/BCG. Trained immunity is epigenetically regulated, resulting in a stronger response upon restimulation. Bone marrow progenitors can get stimulated to produce ‘trained ‘myeloid cells for a prolonged period of time, thereby providing a compelling framework for durable therapeutic interventions.

FIG. 15 is an illustration of a cell showing trained immunity is regulated at the cellular level by bacterial, fungal and metabolic pathways, resulting in epigenetic modifications that undertie cytokine secretion.

FIG. 16 is an illustration of an overview of processes and show bone marrow-avid nanomaterials that either inhibit (green) or promote (red) trained immunity can be employed to prime the immune system and treat a variety of conditions, ranging from cardiovascular disease and its clinical consequences, autoimmune disorders, to sepsis and infections, as well as cancer.

FIG. 17 is an illustration of priming the immune system’s susceptibility toward immune checkpoint blockade therapy can be achieved by promoting trained immunity.

FIG. 18 is a graphic illustration of the radioisotope labeling process.

FIG. 19 is a graphic illustration of PET imaging using a radioisotope delivered by nanobiologic and shows accumulation of the nanobiologic in the bone marrow and spleen of a mouse, rabbit, monkey, and pig model.

### DETAILED DESCRIPTION OF THE INVENTION

The invention is directed to nanobiologic composition for promoting trained immunity, methods of making such nanobiologics, methods of incorporating drug into said nanobiologics, and pro-drug formulations combining drug with functionalized linker moieties such as phospholipids, aliphatic chains. sterols.

Inflammation is triggered by innate immune cells as a defense mechanism against tissue injury. An ancient mechanism of immunological memory, named trained immunity, also called innate immune memory, as defined by a long-term increased responsiveness (e.g. high cytokine production) after re-stimulation with a secondary stimulus of myeloid innate immune cells, being induced by a primary insult stimulating these cells or their progenitors and stem cells in the bone marrow, and mediated by epigenetic, metabolic and transcriptional rewiring.

Trained Immunity is defined by a secondary long-term hyper-responsiveness, as manifested by increased cytokine excretion caused by the metabolic and epigenetic rewiring, to re stimulation after a primary insult of the myeloid cells, the myeloid progenitors, and the hematopoietic stem cells in the bone marrow, blood, and/or spleen.

The invention is directed in one preferred embodiment to a myeloid cell- specific nanoimmunotherapy, based on delivering a nanobiologic carrying or having an incorporated STIMULATOR, which promotes epigenetic and metabolic modifications underlying trained immunity. The invention relates to therapeutic nanobiologic compositions and methods of treating patients who have cancer, by promoting trained immunity, which is the long-term increased responsiveness, the result of metabolic and epigenetic re-wiring of myeloid cells and their stem cells and progenitors in the bone marrow and spleen and blood induced by a primary insult, and characterized by increased cytokine excretion after re- stimulation with one or multiple secondary stimuli.

### Definitions

### Treating or Treatment

The phrase “treating” or “treatment” of a state, disorder or condition includes:
(1) preventing or delaying the appearance of clinical symptoms of the state, disorder, or condition developing in a person who may be afflicted with or predisposed to the state, disorder or condition but does not yet experience or display clinical symptoms of the state, disorder or condition; or
(2) inhibiting the state, disorder or condition, i.e., arresting, reducing or delaying the development of the disease or a relapse thereof (in case of maintenance treatment) or at least one clinical symptom, sign, or test, thereof; or
(3) relieving the disease, i.e., causing regression of the state, disorder or condition or at least one of its clinical or sub-clinical symptoms or signs.

### Nanobiologic

The term “nanobiologic” refers to (i) a nanoscale assembly, having (ii) a promotor drug incorporated in the nanoscale assembly, wherein the drug is an promotor of the inflammasome, an promotor of metabolic pathways, and/or an promotor of epigenetic pathways within a hematopoietic stem cell (HSC), common myeloid progenitor (CMP), or a myeloid cell.

### Nanoscale Assembly

The term “nanoscale assembly” (NA) refers to a multi-component carrier composition for carrying the active payload, e.g. drug. The nanoscale assembly comprises the subcomponents: (a) phospholipids, (b) apolipoprotein A-I (apoA-I) or a peptide mimetic of apoA-I, and optionally (c) a hydrophobic matrix. The nanoscale assembly can also optionally include (d) cholesterol.

The term “nanoscale assembly” (NA) also refers to a multi-component carrier composition comprising: (a) phospholipids, (b) apolipoprotein A-I (apoA-I) or a peptide mimetic of apoA-I, and (c) a hydrophobic matrix comprising one or more triglycerides, fatty acid esters, hydrophobic polymers, and sterol esters. The nanoscale assembly can also optionally include (d) cholesterol.

### Phospholipids

The term “phospholipid” refers to an amphiphilic compound that consists of two hydrophobic fatty acid “tails” and a hydrophilic “head” consisting of a phosphate group. The two components are joined together by a glycerol molecule. The phosphate groups can be modified with simple organic molecules such as choline, ethanolamine or serine. Choline refers to an essential, bioactive nutrient having the chemical formula R-(CH2)2-N-(CH2)4. When a phospho- moiety is R- it is called phosphocholine.

Examples of suitable phospholipids include, without limitation, phosphatidylcholines, phosphatidylethanolamines, phosphatidylinositol, phosphatidylserines, sphingomyelin or other ceramides, as well as phospholipid-containing oils such as lecithin oils. Combinations of phospholipids, or mixtures of a phospholpid(s) and other substance(s), may be used.

Non-limiting examples of the phospholipids that may be used in the present composition include phosphatidylcholines (PC), phosphatidylglycerols (PG), phosphatidylserines (PS), phosphatidylethanolamines (PE), and phosphatidic acid/esters (PA), and lysophosphatidylcholines.

Specific examples include: DDPC CAS-3436-44-0 I,2-Didecanoyl-an-glycero-3-phosphocholine, DEPA-NA CAS-80724-31-8 I,2-Dierucoyl-sn-glycero-3-phosphate (Sodium Salt), DEPC CAS-56649-39-9 I,2-Dierucoyl-sn-glycero-3-phosphocholine, DEPE CAS-988-07-2 I,2-Dierucoyl-sn-glycero-3-phosphoethanolamine, DEPG-NA 1,2-Dierucoyl-sn-glycero-3[Phospho-rac-(I-glycerol...) (Sodium Salt), DLOPC CAS-998-06-1 1,2-Dilinoleoyl-sn-glycero-3-phosphocholine, DLPA-NA 1,2-Dilauroyl-sn-glycero-3-phosphate (Sodium Salt), DLPC CAS-18194-25-7 I,2-Dilauroyl-sn-glycero-3-phosphocholine, DLPE 1,2-Dilauroyl-sn-glycero-3-phosphoethanolamine, DLPG-NA 1,2-Dilauroyl-sn-glycero-3[Phospho-rac-(I-glycerol...) (Sodium Salt). DLPG-NH4 I,2-Dilauroyl-sn-glycero-3[Phospho-rac-(I-glycerol...) (Ammonium Salt), DLPS-NA I,2-Dilauroyl-sn-glycero-3-phosphoserine (Sodium Salt), DMPA-NA CAS-80724-3 I,2-Dimyristoyl-sn-glycero-3-phosphate (Sodium Salt), DMPC CAS-18194-24-6 I,2-Dimyristoyl-sn-glycero-3-phosphocholine, DMPE CAS-988-07-2 I,2-Dimyristoyl-sn-glycero-3-phosphoethanolamine, DMPG-NA CAS-67232-80-8 I,2-Dimyristoyl-sn-glycogo-3[Phospho-rac-(I-glycerol...) (Sodium Salt), DMPG-NH4 I,2-Dimyrisloyl-sn-glycero-3[Phospho-rac-(I-glycerol...) (Ammonium Salt), DMPG-NH4/NA I,2-Dimyristoyl-sn-glycero-3[Phospho-rac-(I-glycerol...) (Sodium/Ammonium Salt), DMPS-NA I,2-Dimyristoyl-sn-glycero-3-phosphoserine (Sodium Salt), DOPA-NA 1,2-Dioleoyl-sn-glycero,3-phosphate (Sodium Salt), DOPC CAS-4235-95-4 I,2-Dioleoyl-sn-glycero-3-phosphocholine, DOPE CAS-4004-5-1 I,2-Dioleoyl-sn-glycero-3-phosphoethanolamine, DOPG-NA CAS-62700-69-0 1.2-Dioleoyl-sn-glycero-3[Phospho-rac-(I-glycerol... )(Sodium Salt), DOPS-NA CAS-70614-14-1 I,2-Dioleoyl-sn-glycero-3-phosphoserine (Sodium Salt), DPPA-NA CAS-71065-87-7 I,2-Dipalmitoyl-sn-glycero-3-phosphate (Sodium Salt), DPPC CAS-63-89-8 1,2-Dipalmitoyl-sn-glycero-3-phosphocholine, DPPE CAS-923-61-5 1,2-Dipalmitoyl-sn-glycero-3-phosphoethanolamine, DPPG-NA CAS-67232-81-9 1,2-Dipalmitoyl-sn-glycero-3(Phospho-rac-(I-glycerol...) (Sodium Salt), DPPG-NH4 CAS-73548-70-6 1,2-Dipalmitoyl-sn-glycero-3[Phospho-rac-(I-glycerol...) (Ammonium Salt), DPPS-NA 1,2-Dipalmitoyl-sn-glycero-3-phosphoserine (Sodium Salt), DSPA-NA CAS-108321-18-2 1,2-Distearoyl-sn-glycero-3-phosphate (Sodium Salt), DSPC CAS-816-94-4 I,2-Distearoyl-sn-glycero-3-phosphocholine, DSPE CAS- 1069-79-0 I,2-Distearoyl-sn-glycero-3-phosphoethanolamine, DSPG-NA CAS-67232-82-0 1,2-Distearoyl-sn-glycero-3 [Phospho-rac-(I -glycerol...)

(Sodium Salt), DSPG-NH4 CAS- 108347-80-4 I,2-Distearoyl-sn-glycero-3[Phospho-rac-(I-glycerol...) (Ammonium Salt), DSPS-NA I,2-Distearoyl-sn-glycero-3-phosphoserine (Sodium Salt), EPC Egg-PC, HEPC Hydrogenated Egg PC, HSPC Hydrogenated Soy PC, LYSOPC MYRISTIC CAS-18194-24-6 I-Myristoyl-sn-glycero-3-phosphocholine, LYSOPC PALMITIC CAS-17364-16-8 I-Palmitoyl-sn-glycero-3-phosphocholine, LYSOPC

STEARIC CAS-19420-57-6 I-Stearoyl-sn-glycero-3-phosphocholine, Milk Sphingomyelin, MPPC I-Myristoyl-2-palmitoyl-sn-glycero 3-phosphocholine, MSPC I-Myristoyl-2-stearoyl-sn-glycero-3-phosphocholine, PMPC I-Palmitoyl-2-myristoyl-sn-glycero-3-phosphocholine, POPC CAS-26853-S1-6 I-Palmitoyl-2-oleoyl-sn-glycero-3-phosphocholine, POPE I-Palmitoyl-2-oleoyl-sn-glycero-3-phosphoethanolamine, POPG-NA CAS-81490-05-3 I-Palmitoyl-2-oleoyl-sn-glycero-3[Phospho-rac-(I-glycerol)...] (Sodium Salt), PSPC 1-Palmiloyl-2-stearoyl-sn-glycero-3-phosphocholine, SMPC 1 -Stearoyl-2-myristoyl-sn-glycero-3-phosphocholine, SOPC I-Stearoyl-2-oleoyl-sn-glycero-3-phosphocholine, SPPC I-Stearoyl-2-palmitoyl-sn-glycero-3-phosphocholine.

In some preferred embodiments, specific non-limiting examples of phospholipids include: dimyristoylphosphatidylcholine (DMPC), soy lecithin, dipalmitoylphosphatidylcholine (DPPC), distearcylphosphatidylcholine (DSPC), dilaurylolyphosphatidylcholine (DLPC), dioleoylphosphatidylcholine (DOPC), dilaurylolylphosphatidylglycerol (DLPG), dimyristoylphosphatidylglycerol (DMPG), dipalmitoylphosphatidylglycerol (DPPG), distearoylphosphatidylglycerol (DSPG), dioleoylphosphatidylglycerol (DOPG), dimyristoyl phosphatidic acid (DMPA), dimyristoyl phosphatidic acid (DMPA), dipalmitoyl phosphatidic acid (DPP A), dipalmitoyl phosphatidic acid (DPP A), dimyristoyl phosphatidylethanolamine (DMPE), dipalmitoyl phosphatidylethanolamine (DPPE), dimyristoyl phosphatidylserine (DMPS). dipalmitoyl phosphatidylserine (DPPS), dipalmitoyl sphingomyelin (DPSP), distearoyl sphingomyelin (DSSP), and mixtures thereof.

In certain embodiments, when the present composition comprises (consists essentially of, or consists of) two or more types of phospholipids, the weight ratio of two types of phospholipids may range from about 1:10 to about 10:1, from about 2:1 to about 4:1, from about 1 :1 to about 5:1, from about 2:1 to about 5:1, from about 6:1 to about 10:1, from about 7:1 to about 10:1, from about 8:1 to about 10:1, from about 7:1 to about 9:1, or from about 8:1 to about 9:1. For example, the weight ratio of two types of phospholipids may be about 1:10, about 1:9, about 1:8, about 1:7, about 1:6, about 1:5, about 1:4, about 1:3, about 1:2, about 1 :1, about 2:1, about 3:1, about 4:1, about 5:1, about 6:1, about 7:1, about 8:1, about 9:1, or about 10:1.

In one embodiment, the (a) phospholipids of the present nanoscale assembly comprise (consist essentially of, or consist of) a mixture of a two-chain diacyl- phospholipid and a single chain acyl-phospholipid/lysolipid.

In one embodiment, the (a) phospholipids is a mixture of phospholipid and lysolipid is (DMPC), and (MHPC).

The weight ratio of DMPC to MHPC may range from about 1:10 to about 10:1, from about 2:1 to about 4:1, from about 1:1 to about 5:1, from about 2:1 to about 6:1, from about 6:1 to about 10:1, from about 7:1 to about 10:1, from about 8:1 to about 10:1, from about 7:1 to about 9:1, or from about 8:1 to about 9:1. The weight ratio of DMPC to MHPC may be about 1:10, about 1:9, about 1:8, about 1:7, about 1:6, about 1:5, about 1:4, about 1:3, about 1:2, about 1 :1, about 2:1, about 3:1, about 4:1, about 5:1, about 6:1, about 7:1, about 8:1, about 9:1, or about 10:1.

In one embodiment, the (a) phospholipids is a mixture of phospholipid and lysolipid is (POPC) and (PHPC).

The weight ratio of POPC to PHPC may range from about 1 :10 to about 10:1, from about 2: 1 to about 4:1, from about 1:1 to about 5:1, from about 2:1 to about 5:1, from about 6:1 to about 10:1, from about 7:1 to about 10:1, from about 8:1 to about 10:1, from about 7:1 to about 9:1, or from about 8:1 to about 9:1. The weight ratio of POPC to PHPC may be about 1:10, about 1:9, about 1:8, about 1:7, about 1:6, about 1:5, about 1:4, about 1:3, about 1:2, about 1 :1, about 2:1. about 3:1, about 4:1, about 5:1, about 6:1, about 7:1, about 8:1, about 9:1, or about 10:1.

It is noted that all phospholipids ranging in chain length from C4 to C30, saturated or unsaturated, cis or trans, unsubstituted or substituted with 1-6 side chains, and with or without the addition of lysolipids are contemplated for use in the nanoscale assembly or nanoparticles/nanobiologics described herein.

Additionally, other synthetic variants and variants with other phospholipid headgroups are also contemplated.

“Lysolipids” as used herein, include (acyl-, single chain) such as in non-limiting embodiments I-myristoyl-2-hydroxy-sn-glycero-3-phosphocholine (MHPC), I-Palmitoyl-2-hexadecyl-sn-glycero-3-phosphocholine (PHPC) and I-stearoyl-2-hydroxy-sn-glycero-3-phosphocholine (SHPC).

### Apolipoprotein A-I (apoA-I) (apoAI)

The term “apolipoprotein A-I” or “apoA-I”, and also “apoliprotein AI” or “apoAI”, refers to a protein that is encoded by the APQA1 gene In humans, and as used herein also includes peptide mimetics of apoA-I. Apolipoprotein Al (spoA-I) is subcomponent (b) in the nanoscale assembly.

### Hydrophobic Matrix

The term “hydrophobic matrix” refers to a core or filler or structural modifier of the nanobiologic. Structural modifications include (1) using the hydrophobic matrix to increase or design the particle size of a nanoscale assembly made from only (a) phospholipids and (b) apoA-l. (2) Increasing or decreasing (designing) the rigidity of the nanoscale assembly particles, (3) increasing or decreasing (designing) the viscosity of the nanoscale assembly particles, and (4) increasing or decreasing (designing) the biodistribution characteristics of the nanoscale assembly particles.

Nanoscale assembly particle size, ngidity, viscosity, and/or biodistribution, can be moderated by the quantity and type of hydrophobic molecule added. In a non-limiting example, a nanoscale assembly made from only (a) phospholipids and (b) apoA-l may have a diameter of 10 nm-50 nm. Adding (c) a hydrophobic matrix molecule such as triglycerides, swells the nanoscale assembly from a minimum of IOnm to at least 30 nm. Adding more triglycerides can increase the diameter of the nanoscale assembly to at least 50 nm, at least 75rim, at least IOOnm, at least 50 nm, at least 200 nm, at least 300 nm, and up to 400 nm within the scope of the invention.

Production methods can prepare uniform size nanoscale assembly particles, or a non-uniform sized mixture of nanoscale assembly particles, either by not filtering, or by prepanng a range of different sized nanoscale assembly particles and re-combining them in a post-production step. The larger the size of the nanoscale assembly particles, the more drug can be Incorporated. However, larger sizes e.g. >120 nm, can limit, prevent or slow diffusion of the nanoscale assembly particles into the tissues of the patient being treated. Smaller nanoscale assembly particles do not hold as much drug per particle, but are able to access the bone marrow, blood, or spleen, or other localized tissue affected by trained immunity, e.g. transplant and surrounding tissues, atherosclerotic plaque, and so forth (biodistribution). Using a non-uniform mixture of nanoparticles sizes in a single administration or regimen can produce an Immediate reduction in innate immune hyper-responsiveness, and simultaneously produce a durable, long-term reduction in innate immune hyper- responsiveness that can last days, weeks, months, and years, wherein the nanobiologic has reversed, modified, or re regulated the metabolic, epigenetic, and inflammasome pathways of the hematopoietic stem cells (HSC), the common myeloid progenitors (CMP), and the myeloid cells such as monocytes, macrophages and other short-lived circulating cells.

Adding other (c) hydrophobic matrix molecules, such as cholesterol, fatty acid esters, hydrophobic polymers, sterol esters, and different types of triglycerides, or specific mixtures thereof, can further design the nanoscale assembly particles to emphasize specific desired characteristics for specific purposes. Size, rigidity, and viscosity can affect loading and biodistribution.

By way of non-limiting example, maximum loading capacity can be determined dividing the Volume of the interior of the nanoscale assembly particle by the Volume of a drug-load spheroid.

Particle: assume a IOOnm spherical particle having 2.2 nm-3.0 nm phospholipid wall, yielding a 94 nm diameter interior with Volume (L) @ 413 i(r)3.

Drug: assume STIMULATOR at 12x12x35 Angstrom or as a cylinder 1.2x1.2x3.5 nm, where multiple drug molecule cylinders, e.g. seven or nine, etc. could assume a 3.5 nm diameter spheroid having a radius of 1.75 nm VoI(small) @ 4/3 i(r)3.

Maximum Loading Capacity (calc): ..487k 3.5 nm spheroids within a IOOnm particle.

Biologically relevant lipids include fatty acyls, glycerolipids, glycerophospholipids, sphingolipids, sterol lipids, prenol lipids, saccharolipids, and polyketides. A complete list of over 42,000 lipids can be obtained at https://www.lipidmaps.org.

### Triglyceride

The term Triglyceride″ and like terms mean an ester derived from glycerol and three fatty acids. The notation used in this specification to describe a triglyceride is the same as that used below to describe a fatty acid. The triglyceride can comprise glycerol with any combination of the following fatty acids: 08:1, 04: 1, 06: ₁, polyunsaturated, and saturated. Fatty acids can attach to the glycerol molecule in any order, e.g., any fatty acid can react with any of the hydroxyl groups of the glycerol molecule for forming an ester linkage. Triglyceride of 08:1 fatty acid simply means that the fatty acid components of the triglyceride are derived from or based upon a 08:1 fatty acid. That is, a 08:1 triglyceride is an ester of glycerol and three fatty acids of 18 carbon atoms each with each fatty acid having one double bond. Similarly, a 04:1 triglyceride is an ester of glycerol and three fatty acids of 14 carbon atoms each with each fatty acid having one double bond. Likewise, a 06:1 triglyceride is an ester of glycerol and three fatty acids of 18 carbon atoms each with each fatty acid having one double bond. Triglycerides of 08:1 fatty acids in combination with 04:1 and/or 06:1 fatty acids means that: (a) a 08:1 triglyceride Is mixed with a 04:1 triglyceride or a 06: 1 triglyceride or both; or (b) at least one of the fatty acid components of the triglyceride is derived from or based upon a 08:1 fatty acid, while the other two are derived from or based upon 04:1 laity acid and/or 06:1 fatty acid.

### Fatty Acid

The term “fatty acid” and like terms mean a carboxylic acid with a long aliphatic tail that is either saturated or unsaturated. Fatty acids may be esterified to phospholipids and triglycerides. As used herein, the fatty acid chain length includes from C4 to C30, saturated or unsaturated, cis or trans, unsubstituted or substituted with 1-6 side chains. Unsaturated fatty acids have one or more double bonds between carbon atoms. Saturated fatty acids do not contain any double bonds. The notation used in this specification for describing a fatty acid includes the capital letter ‘C’ for carbon atom, followed by a number describing the number of carbon atoms in the fatty acid, followed by a colon and another number for the number of double bonds in the fatty acid. For example, Cl 6:1 denotes a fatty acid of 16 carbon atoms with one double bond. e.g., palmitoleic acid. The number after the colon in this notation neither designates the placement of the double bond(s) in the fatty acid nor whether the hydrogen atoms bonded to the carbon atoms of the double bond are cis to one another. Other examples of this notation include 08:0 (stearic acid), 08:1 (oleic acid), 08:2 (linoleic acid), 08:3 (a- linolenic acid) and C20:4 (arachidonic acid).

### Sterols and Sterol Esters

The term “sterols” such as, but not limited to cholesterol, can also be utilized in the methods and compounds described herein. Sterols are animal or vegetable steroids which only contain a hydroxyl group but no other functional groups at C-3. In general, sterols contain 27 to 30 carbon atoms and one double bond in the 5/6 position and occasionally in the 7/8, 8/9 or other positions. Besides these unsaturated species, other sterols are the saturated compounds obtainable by hydrogenation. One example of a suitable animal sterol is cholesterol. Typical examples of suitable phytosterols, which are preferred from the applicational point of view, are ergosterols, campesterols, stigmasterols, brassicasterols and, preferably, sitosterols or sitostanols and, more particularly, b-sitosterols or b-sitostanols. Besides the phytosterols mentioned, their esters are preferably used. The acid component of the ester may go back to carboxylic acids corresponding to formula (I):

R1C0- OH (I)

in which R1C0 is an aliphatic, linear or branched acyl group contaning 2 to 30 carbon atoms and 0 and/or 1, 2 or 3 double bonds. Typical examples are acetic acid, propionic acid, butyric acid, valeric acid, caproic acid, caprylic acid, 2-ethyl hexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, conjugated linoleic acid (CL A), linolenic acid, elaeosteric add, arachic acid, gadoleic acid, behenic acid and erucic acid.

### Hydrophobic Polymers

The hydrophobic polymer or polymers used to make up the matrix may be selected from the group of polymers approved for human use (i.e. biocompatible and FDA-approved).

Such polymers comprise, for example, but are not limited to the following polymers, derivatives of such polymers, co-polymers, block co-polymers, branched polymers, and polymer blends: polyalkenedicarboxlates, polyanhydrides, poly(aspartic acid), polyamides, polybutylenesuccinates (PBS), polybutylenesuccinates-co-adipate (PBSA), poly(e-caprolactone) (PCL), polycarbonates including poly-alkylene carbonates (PC), polyesters including aliphatic polyesters and polyester-amides, polyethylenesuccinates (PES), polyglycolides (PGA), polyimines and polyalkyleneimines (PI, PAI), polylactides (PLA, PLLA, PDLLA), polylactic-co-glycolic acid (PLGA), poly (l-ly sine), polymethacrylates, polypeptides, poly orthoesters, poly-p-dioxanones (PPDO), (hydrophobic) modified-polysaccharides, polysiloxanes and poly-alkyl-siloxanes, polyureas, polyurethanes, and polyvinyl alcohols.

### Prodrug

As used herein and unless otherwise indicated, the term″prodrug” means a derivative of a compound that can hydrolyze, oxidize, or otherwise react under biological conditions (in vitro or in vivo ) to provide the compound. Examples of prodrugs include, but are not limited to, derivatives of nanobiologic composition of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of nanobiologic composition of the Invention that comprise- NO. - NO2, - ONO, or- ONO2 moieties. Prodrugs can typically be prepared using well-known methods, such as those described in 1 Burger’s Medicinal Chemistry and Drug Discovery, 172-178, 949-982 (Manfred E. Wolff ed., 5th ed. 1995), and Design of Prodrugs (H. Bundgaard ed.. Elselvier, N.Y. 1985).

Increasing a drug’s compatibility with nanobiologics can be achieved using the strategy described below. A drug is covalently coupled to a hydrophobic moiety, such as cholesterol. If required, a prodrug approach can be achieved via a labile conjugation, resulting in e.g., an enzymatically cleavable prodrug.

Subsequently, the derivatized drug is incorporated into lipid based nanobiologics used for in vivo drug delivery. The main goal of the drug derivatization is to form a drug-conjugate with a higher hydrophobicity as compared to the parent drug. As a result, the retention of the drug-conjugate inside the nanobiologic is enhanced compared to that of the parent drug, thereby resulting in reduced leakage and improved delivery to the target tissue. In case of the prodrug strategy, different type of hydrophobic moieties might give rise to different in vivo cleavage rates, thereby influencing the rate with which the active drug is generated, and thus the overall therapeutic effect of the nanobiologic-drug construct.

### Biohydrolyzable

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxy ethyl esters), lactonyl esters (such as phthalidyl and thiophthalidyl esters), lower alkoxyacyloxyalkyl esters (such as methoxycarbonyl-oxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, a-amino acid amides, alkoxyacyl amides, and alkylaminoalkylcarbonyl amides. Examples of biohydrolyzable carbamates include, but are not limited to lower alkylamines, substituted ethylenediamines, amino acids, hydroxyalkylamines, heterocyclic and heteroaromatic amines, and poly ether amines.

### Methods of Producing the Nanoscale Assembly

Methods are described below, and there are variations relating to these methods.

### Method 1.

A. The phospholipids, (pro-)drug and optional triglycerides or polymer are dissolved (typically in chloroform, ethanol or acetonitrile). This solution is then evaporated under vacuum to form a film of the components. Subsequently, a buffer solution is added to hydrate the film and generate a vesicle suspension.

B. The phospholipids, (pro-)drug and optional triglycerides or polymer are dissolved (typically in chloroform, ethanol or acetonitrile). This solution is infused- or added drop-wise-to a mildly heated buffer solution under stirring, until complete evaporation of the organic solvents, generating a vesicle suspension.

To the vesicle suspension, generated using A or B, apolipoprotein A-1 (apoA-l) (note that apoA-I can also already be in B) - use dropwise to avoid denature, is added and the resulting mixture is sonicated for 30 minutes using a tip sonicator while being thoroughly cooled using an external ice-water bath. The obtained solution containing the nanobiologics and other by products is transferred to a Sartorius Vivaspin tube with a molecular weight cut-off depending on the estimated size of the nanobiologics (typically Vivaspin tubes with cut-offs ol 10.000-100.000 kDa are used). The tubes are centrifuged until ∼90% of the solvent volume has passed through the filter. Subsequently, a volume of buffer, roughly equal to the volume of the remaining solution, is added and the tubes are spun again until roughly half the volume has passed through the filter. This is repeated twice after which the remaining solution is passed through a polyethersultone 0.22 pm syringe filter, resulting in the final nanobiologic solution.

### Method 2.

In an alternative approach, the phospholipids, (pro-)drug and optional triglycerides or polymer are dissolved (typically in ethanol or acetonitrile) and loaded into a syringe.

Additionally, a solution of apolipoprotein A-l (apoA-1) in phosphate buffered saline is loaded into a second syringe. Using microfluidics pumps, the content of both syringes is mixed using a microvortex platform. The obtained solution containing the nanobiologics and other by products is transferred to a Sartorius Vivaspin tube with a molecular weight cut-off depending on the estimate size of the particles (typically Vivaspin tubes with cut-offs of 10.000-100.000 kDa are used). The tubes are centrifuged until ~90% of the solvent volume has passed through the filter. Subsequently, a volume of phosphate buffered saline roughly equal to the volume of the remaining solution is added and the tubes are spun again until roughly half the volume has passed through the filter. This is repeated twice after which the remaining solution is passed through a polyethersulfone 0.22 pm syringe filter, resulting in the final nanobiologic solution.

### Microfluidizer Method

In another preferred method according to the invention, microfluidizer technology is used to prepare the nanoscale assembly and the final nanobiologic composition.

Microfluidizers are devices for preparing small particle size materials operating on the submerged jet principle. In operating a microfluidizer to obtain nanoparticulates, a premix flow is forced by a high pressure pump through a so-called interaction chamber consisting of a system of channels in a ceramic block which split the premix into two streams. Precisely controlled shear, turbulent and cavitational forces are generated within the interaction chamber during microfluidization. The two streams are recombined at high velocity to produce shear. The so-obtained product can be recycled into the microfluidizer to obtain smaller and smaller particles. Advantages of microfluidization over conventional milling processes include substantial reduction of contamination of the final product, and the ease of production scaleup.

### Combination Therapy - Nanobiologic Delivery With Checkpoint Inhibitors

Also contemplated as within the scope of the present inventive subject matter are checkpoint inhibitors and combination treatments with trained immunity-inducing nanobiologics.

### Checkpoint Inhibitor

A checkpoint inhibitor refers to a type of drug that blocks certain proteins made by some types of immune system cells, such as T cells, and some cancer cells. These proteins help keep immune responses in check and can keep T cells from killing cancer cells. When these proteins are blocked, the“brakes” on the immune system are released and T cells are able to kill cancer cells better. Examples of checkpoint proteins found on T cells or cancer cells Include PD-1/PD-L1 and CTLA-4/B7-1/B7-2. Some immune checkpoint inhibitors are used to treat cancer.

### Checkpoint Inhibitor Background

Immune checkpoints regulate T cell function in the immune system. T cells play a central role in cell-mediated immunity. Checkpoint proteins interact with specific ligands which send a signal to the T cell and essentially turn off or inhibit T cell function. Cancer cells take advantage of this system by driving high levels of expression of checkpoint proteins on their surface which results in control of the T cells expressing checkpoint proteins on the surface of T cells that enter the tumor microenvironment, thus suppressing the anticancer immune response. As such, inhibition of checkpoint proteins results in complete or partial restoration of T cell function and an immune response to the cancer cells. Examples of checkpoint proteins include, but are not limited to CTLA-4, PD-L1, PD-L2, PD-I, B7-H3, B7- H4, BTLA, HVEM, TIM3, GAL9, LAG3, VISTA, KIR, 2B4 (belongs to the CD2 family of molecules and is expressed on all NK, gd, and memory CD8+ (ab) T cells), CD 160 (also referred to as BY55), CGEN- 15049, CHK 1 and CHK2 kinases, A2aR and various B-7 family ligands.

### Types of Checkpoint Inhibitors

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands.

Illustrative checkpoint molecules that may be targeted for blocking or inhibition to re-activate the immune response include, but are not limited to, CTLA-4, PD-L1, PD-L2, PD-I, B7-H3, B7-H4, BTLA, HVEM, GAL9, LAG3, TIM3, VISTA, KIR, 2B4 (belongs to the CD2 family of molecules and is expressed on all NK, gd, and memory CD8+ (ab) T cells), CD 160 (also referred to as BY55), CGEN- 15049, CHK 1 and CHK2 kinases, A2aR and various B-7 family ligands. B7 family ligands include, but are not limited to, B7-1, B7-2, B7-DC, B7-H1, B7-H2, B7-H3, B7-H4, B7-H5, B7-H6 and B7-H7.

Checkpoint inhibitors include antibodies, or antigen binding fragments thereof, other binding proteins, biologic therapeutics or small molecules, that bind to and block or inhibit the activity of one or more of CTLA-4, PD-L1, PD-L2, PD-I, BTLA, HVEM, TIM3, GAL9, LAGS, VISTA. KIR, 2B4, CD 160 and CGEN- 15049.

Illustrative immune checkpoint inhibitors include Tremelimumab (CTLA-4 blocking antibody), anti-OX40, PD-L1 monoclonal Antibody (anti-B7-Hl: MEDI4736), MK-3475 (PD-1 blocker), Nivolumab (anti-PDI antibody). CT- 011 (anti-PDI antibody). BY55 monoclonal antibody, AMP224 (anti-PDLI antibody), BMS- 936559 (anti-PDLI antibody), MPLDL3280A (anti-PDLI antibody), MSB0010718C (anti- PDL1 antibody) and Yervoy/ipilimumab (anti-CTLA-4 checkpoint inhibitor). Checkpoint protein ligands include, but are not limited to PD-L1, PD-L2, B7-H3, B7-H4, CD28, CD86 and TIM-3.

Checkpoint inhibitors that block PD-1 include nivolumab (Opdivo), and pembrolizumab (Keytruda). Nivolumab and pembrolizumab are treatments for some people with melanoma skin cancer, Hodgkin lymphoma, non-small cell lung cancer, and cancer of the urinary tract (urothelial cancer). The urinary tract includes the center of the kidney (renal pelvis), the tubes that take urine from the kidneys to the bladder (ureters), the bladder, and the tube that drains urine from the bladder and out of the body (urethra)

Checkpoint inhibitors that block CTLA-4 include lpilimumab (Yervoy), which is used as a treatment for advanced melanoma.

Checkpoint inhibitors that block PD-L1 include atezolizumab (also known as MPDL3280A). Atezolizumab is a treatment for some people with lung cancer and urothelial cancers. It is also in clinical trials for other cancers including breast cancer.

Programmed cell death protein 1 (PD-I) is a 288 amino acid cell surface protein molecule expressed on T cells and pro-B cells and plays a role in their fate/ differentiation. PD-I has two ligands, PD-L1 and PD-L2, which are members of the B7 family. PD-I plays a role in tumor-specific escape from immune surveillance. PD-1 is up-regulated in melanoma infiltrating T lymphocytes (TILs) (Doth (2009) Blood 114 (8): 1457-58). Tumors have been found to express the PD-I ligand (PDL-1 and PDL-2) which, when combined with the up-regulation of PD-I in CTLs, may be a contributory factor in the loss in T cell functionality and the inability of CTLs to mediate an effective anti-tumor response.

Clinical trials in melanoma have shown robust anti- tumor responses with anti-PD-I blockade. Significant benefit with PD-I inhibition in cases of advanced melanoma, ovarian cancer, non-small-cell lung, prostate, renal-cell, and colorectal cancer have also been described Studies in murine models have applied this evidence to glioma therapy. Anti-PD-1 blockade adjuvant to radiation promoted cytotoxic T cell population and an associated long-term survival benefit in mice with glioma tumor.

In view of the results provided herein, an aspect of the present disclosure includes combined treatment of any solid tumor with any checkpoint inhibitor in combination with one or more of a trained immunity-inducing nanobiologic such as MDP-HDL, MTP-HDL. PG-HDL, BG-HDL, and UA-HDL.

### Antibody Checkpoint Inhibitors

One aspect of the present disclosure provides checkpoint inhibitors which are antibodies that can act as inhibitors of PD-I, thereby modulating immune responses regulated by PD-I, In one embodiment, the anti-PD-I antibodies can be antigen-binding fragments. Anti-PD-I antibodies disclosed herein are able to bind to human PD-I and agonize the activity of PD-1, thereby inhibiting the function of immune cells expressing PD-1. Examples of PD-I and PD-U blockers are described in U.S. Pat. Nos. 7,488,802; 7,943,743; 8,008,449; 8,168,757; 8,217,149, and PCT Published Patent Application Nos: W003042402, WO2008156712, W02010089411, W02010036959, WO2011066342, WO2011159877, WO2011082400, and WO2011161699.

There are several PD-1 inhibitors currently being tested in clinical trials. CT-011 is a humanized IgGl monoclonal antibody against PD-I, A phase II clinical trial in subjects with diffuse large B-cell lymphoma (DLBCL) who have undergone autologous stem cell transplantation was recently completed. Preliminary results demonstrated that 70% of subjects were progression-free at the end of the follow-up period, compared with 47% in the control group, and 82% of subjects were alive, compared with 62% in the control group. This trial determined that CT-011 not only blocks PD-1 function, but it also augments the activity of natural killer cells, thus intensifying the antitumor immune response.

BMS 936558 is a fully human IgG4 monoclonal antibody targeting PD-1. In a phase I trial, biweekly administration of BMS-936558 in subjects with advanced, treatment-refractory malignancies showed durable partial or complete regressions. The most significant response rate was observed in subjects with melanoma (28%) and renal cell carcinoma (27%), but substantial clinical activity was also observed in subjects with non- small cell lung cancer (NSCLC), and some responses persisted for more than a year.

BMS 936559 is a fully human IgG4 monoclonal antibody that targets the PD-1 ligand PD-L1. Phase I results showed that biweekly administration of this drug led to durable responses, especially in subjects with melanoma. Objective response rates ranged from 6% to 17%) depending on the cancer type in subjects with advanced-stage NSCLC, melanoma, RCC, or ovarian cancer, with some subjects experiencing responses lasting a year or longer MK 3475 is a humanized IgG4 anti- PD-1 monoclonal antibody in Phase III study alone or in combination with chemotherapy versus chemotherapy alone as first-line therapy for advanced gastric or gastroesophageal junction (GEJ) adenocarcinoma. MK 3475 is currently undergoing numerous global Phase III clinical trials.

MPDL 3280A (atezolizumab) is a monoclonal antibody, which also targets PD-L1. MPDL 3280A received Breakthrough Therapy Designation from the U.S. Food and Drug Administration (FDA) for the treatment of people whose NSCLC expresses PD-L1 and who progressed during or after standard treatments.

AMP 224 is a fusion protein of the extracellular domain of the second PD-1 ligand, PD-L2, and IgGl, which has the potential to block the PD-L2/PD- 1 interaction. AMP-224 is currently undergoing phase I testing as monotherapy in subjects with advanced cancer.

Medi 4736 is an anti-PD-LI antibody that has demonstrated an acceptable safety profile and durable clinical activity in this dose-escalation study. Expansion in multiple cancers and development of MED 14736 as monotherapy and in combination is ongoing.

Thus, in certain embodiments, the PD-1 blockers include anti-PD-1 antibodies and similar binding proteins such as nivolumab (MDX 1106, BMS 936558, ONO 4538), a fully human lgG4 antibody that binds to and blocks the activation of PD-I by its ligands PD-L1 and PD-L2; pembrolizumab/lambrolizumab (MK-3475 or SCH 900475), a humanized monoclonal IgG4 antibody against PD-I; CT-011 a humanized antibody that binds PD-1; AMP-224 is a fusion protein of B7-DC; an antibody Fc portion; BMS-936559 (MDX- 1105-01) for PD-L1 (B7-H1) blockade. Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94).

### Combination Therapy - Nanobiologic Delivery With Anti-Cancer Agents

Examples of anti-cancer agents include, but are not limited to: acivicin: aclarubicin; acodazole hydrochloride; acronine; adozelesin; aldesleukin; altretamine; ambomycin; ametantrone acetate; amsacrine; anastrozole; anthramycin: asparaginase; asperin: azacitidine; azetepa; azotomycin: batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol, celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin, doxorubicin hydrochloride; droloxifene; droloxifene citrate; dromostanolone propionate; duazomycin; edatrexate; eflomithine hydrochloride: elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine: fadrozole hydrochloride; fazarabine: fenretinide; floxuridine: fludarabine phosphate; fluorouracil; flurocitabine; fosquidone; fostriecin sodium; gemcitabine, gemcitabine hydrochloride; hydroxyurea; idarubicin hydrochloride; ifostamide; ilmofosine: iproplatin; irinotecan; irinotecan hydrochloride; lanreotide acetate; letrozole; leuprolide acetate; liarozole hydrochloride; lometroxol sodium; lomustine; losoxantrone hydrochloride, masoprocol; maytansine; mechlorethamine hydrochloride; megestrol acetate; melengestrol acetate: melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; satingol; safingol hydrochloride; semustine; simtrazene; sparfosate sodium; sparsomycin; spirogermanium hydrochloride; spiromustine; spiroplatin, streptonigrin; streptozocin; sulofenur; talisomycin; tecogalan sodium; taxotere; tegafur; teloxantrone hydrochloride; temoportin; teniposide; teroxirone; testolactone; thiamiprine; thioguanine; thiotepa; tiazofurin; tirapezamine; toremifene citrate; trestolone acetate; triciribine phosphate; trimetrexate; trimetrexate glucuronate; triptorelin; tubulozole hydrochloride; uracil mustard; uredepa; vapreotide; verteportin; vinblastine sulfate; vincristine sulfate: vindesine; vindesine sulfate; vinepidine sulfate; vinglycinate sulfate; vinleurosine sulfate, vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelosin: aldesleukin; ALL-TK antagonists; altretamine; ambamusline; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein- 1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimu stine; axinastatin 1; axinastatin 2; axinastatin 3; azasetron; azatoxin, azatyrosine; baccatin III derivatives; belanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide- amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomitens analogues; clotrimazole; collismycin A; collismycin B; combretastatin A4; combretastatin analogue; conagenin; crambescidin 818; crisnatol; cryptophycin 8; cryptophycin A derivatives: curacin A; cyclopentanthraquinones: cycloplatam; cypemycin, cytarabine octostate; cytolytic factor; cytostatin; dacliximab; decitabine; dehydrodidemnin B; deslorelin; dexamethasone; dexifosfamide; dexrazoxane; dexverapamil; diaziquone; didemnin B; didox: die thy Inor spermine; dihydro-5-azacytidine; dihydrotaxol. 9-; dioxamycin; diphenyl spiromustine; docetaxel; docosanol; dolasetron; doxifluridine; doxorubicin; droloxifene; dronabinol; duocarmycin SA; ebselen; ecomustine; edettosine; edrecolomab; eflomithine, elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride: flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid, idarubicin; idoxifene; idramantone; ilmotosine; ilomastat; imatinib (e.g., Gleevec®), imiquimod: immunostimulant peptides: insulin-like growth factor- 1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F: lamellarin-N triacetate; lanreotide; leinamycin; lenograstim; lentinan sulfate; leptolstatin; letrozole; leukemia inhibiting factor; leukocyte alpha Interferon; leuprolide+estrogen+progesterone; leuprorelin; levamisole, liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutatium texaphyrin; lysofylline; lytic peptides; maitansine; mannostatin A: marimastat; masoprocol; maspin: matrilysin inhibitors; matrix metalloproteinase inhibitors; menogaril; merbarone; meterelin; methioninase; metoclopramide; MlF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim, Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); 06-benzylguanine; octreotide; okicenone; oligonucleotides; onapristone; ondansetron; ondansetron: oracin: oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfostamide; perillyl alcohol; phenazinomycin; phenylacetate; phosphatase inhibitors; picibanil; pilocarpine hydrochionde; pirarubicin: piritrexim; placetin A; placetin B; plasminogen activator inhibitor; platinum complex; platinum compounds; platinum-triamine complex; portimer sodium; porfiromycin: prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; rat antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; Rll retinamide; rohitukine; romurtide; roquinimex; rubiginone Bl; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate, sodium phenylacetate; solverol; somatomedin binding protein; sonermin; sparfosic acid; spicamycin D; spiromustine; splenopentin; spongistatin 1; squalamine; stiplamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; tomoporfin; teniposide; tetrachlorodecaoxide; tetrazomine; thaliblastine; thiocor aline; thrombopoletin; thrombopoietin mimetic; thymalfasin; thymopoietin receptor agonist; thymotrinan; thyroid stimulating hormone; tin ethyl etiopurpurin; tirapazamine; titanocene bichloride: topsentin; toremifene; translation inhibitors; tretinoin; triacetyluridine; triciribine; trimetrexate; triptorelin; tropisetron, turosteride; tyrosine kinase inhibitors; tyrphostins; UBC inhibitors; ubenimex; urogenital sinus-derived growth inhibitory factor; urokinase receptor antagonists; vapreotide: variolin B; velaresol; veramine; verdins; verteporfin; vinorelbine; vinxaltine; vitaxin; vorozole; zanoterone; zeniplatin; zilascorb; and zinostatin stima 1 amer.

### Small Molecule Secondary Agents

Small molecule drugs that can be used in combination therapy with the nanobiologics of the present invention include acetaminophen, acetylsalicylic acid, adriamycin, azathioprine, biaxin, bisphosphonate, busulphan, capacitabine, carboplatin, celecoxib, chloroquine, cisplatinum, cyclophosphamide, cyclosponne, cytarabine, d-penicilamine, decarbazine, daunorubicin, dexamethasone, diflunisal, docetaxel, doxorubicin estramustine sodium phosphate, etoposide, etoricoxib, fenoprofen, fludarabine, flufenamic acid, fluorouracil, flurbiprofen, ganciclovir, gemcitabine, gliadel, GM-CSF, hydroxychloroquine ibuprofen, IL-2, indomethacin, interferon alpha, irinotecan, ketoprofen, leflunomide, leucoverin, lumiracoxib, meclofenamate, mefenamic acid, melphalan, methylprednisolone, methotrexate, naproxen, nimesulide, oblimersen, oxaprozin, pacilitaxel, palmitronate, parecoxib, pegylated interferon alpha, phenylbutazone, piroxicam, prednisone, prednisolone, procarbazineremicade, rofecoxib, steroids, sulfasalazine, sulindac, tamoxifen, taxol, taxotere, temodar, temozolomide, tenoxicam, thiotepa, topotecan, valdecoxib, vinblastine, vincristine, vinorelbine, and zoledronic acid.

### Dosing

Dosing will generally be in the range of 5 pg to 100 mg/kg body weight of recipient (mammal) per day and more usually in the range of 5 pg to 10 mg/kg body weight per day. This amount may be given in a single dose per day or more usually in a number (such as two, three, four, five or six) of sub-doses per day such that the total daily dose is the same. An effective amount of a salt or solvate, thereof, may be determined as a proportion of the effective amount of the compound of a nanobiologic which comprises an promotor, wherein the promotor or a pharmaceutically acceptable salt, solvate, poly-morph, tautomer or prodrug thereof, formulated as nanobiologic using the nanoscale assembly (IMPEPi-NA).

### Cancer

As used herein, the term“canœr” includes, but is not limited to, solid tumors and blood bom tumors. The term“cancer” refers to disease of skin tissues, organs, blood, and vessels, including, but not limited to, cancers of the bladder, blood vessels, bone, brain, breast, cervix, chest, colon, endrometrium, esophagus, eye, head, kidney, liver, lymph nodes, lung, mouth, neck, ovaries, pancreas, prostate, rectum, skin, stomach, testis, throat, thyroid, urothelium, and uterus.

Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant giolma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi’s sarcoma, karotype acute myeloblastic leukemia, Hodgkin’s lymphoma, non-Hodgkin’s lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom’s macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma. In a specific embodiment, the cancer is metastatic. In another embodiment, the cancer is refractory or resistance to chemotherapy or radiation; in particular, refractory to thalidomide.

### General Pharmaceutical Definitions

As used herein, a “prophylactically effective” amount is an amount of a substance effective to prevent or to delay the onset of a given pathological condition in a subject to which the substance is to be administered. A prophytaclically effective amount refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

As used herein, a “therapeutically effective” amount is an amount of a substance effective to treat, ameliorate or lessen a symptom or cause of a given pathological condition in a subject suffering therefrom to which the substance is to be administered.

In one embodiment, the therapeutically or prophylactically effective amount is from about 1 mg of agent/kg subject to about 1 g of agent/kg subject per dosing. In another embodiment, the therapeutically or prophylactically effective amount is from about 10 mg of agent/kg subject to 500 mg of agent/subject. In a further embodiment, the therapeutically or prophylactically effective amount is from about 50 mg of agent/kg subject to 200 mg of agent/kg subject. In a further embodiment, the therapeutically or prophylactically effective amount is about 100 mg of agent/kg subject. In still a further embodiment, the therapeutically or prophylactically effective amount is selected from 50 mg of agent/kg subject, 100 mg of agent/kg subject, 150 mg of agent/kg subject, 200 mg of agent/kg subject, 250 mg of agent/kg subject, 300 mg of agent/kg subject, 400 mg of agent/kg subject and 500 mg of agent/kg subject.

Pharmaceutical compositions of the present invention may be adapted for administration by any appropriate route, for example by the oral (including buccal or sublingual), inhaled, nasal, ocular, or parenteral (including intravenous and intramuscular) route. Such compositions may be prepared by any method known in the art of pharmacy, for example by bringing into association the active ingredient with the carrier(s) or excipient(s). Parenteral dosage forms are preferred.

Parenteral dosage forms can be administered to patients by various routes including, but not limited to, subcutaneous, intravenous (including bolus injection), Intramuscular, and intraarterial. Because their administration typically bypasses patients’ natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended In a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringers Injection, Dextrose Injection, Dextrose and Sodium Chloride Injection, and Lactated Ringer’s injection; water-miscible vehicles such as, but not limited to, ethyl alcohol, polyethylene glycol, and polypropylene glycol; and non-aqueous vehicles such as, but not limited to, com oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention. For example, cyclodextrin and its derivatives can be used to increase the solubility of a nanoscale particle of the invention and its derivatives.

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to Improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### Radiolabelling for Pet Imaging of Accumulation of Drug Within the Body

In a non-limiting preferred embodiment of the invention, there is provided radiopharmaceutical compositions and methods of radiopharmaceutical imaging an accumulation of a nanobiologic within bone marrow, blood, and/or spleen, of a patient affected by trained immunity, comprising.
(i) administering to said patient a nanobiologic composition in an amount effective to promote a hyper-responsive innate immune response,
   wherein the nanobiologic composition comprises (i) a nanoscale assembly, having (ii) a promoter drug incorporated in the nanoscale assembly, and (iii) a position emission tomography (PET) imaging agent incorporated in the nanoscale assembly,
   wherein the nanoscale assembly is a multi-component carrier composition comprising: (a) phospholipids, and, (b) apoA-1 or a peptide mimetic of apoA-1, and optionally (c) a hydrophobic matrix comprising one or more triglycerides, fatty acid esters, hydrophobic polymers, or sterol esters, or a combination thereof, and optionally (d) cholesterol, wherein the promoter drug is a molecular structure that activates or binds to the pathogen recognizing receptors Dectin-1 or NOD2 to induce trained immunity in myeloid cells and their stem cells and progenitors in the bone marrow, blood and spleen, wherein the molecular structures that activate or bind to Dectin- 1 include, but are not limited to, b-glucans and its derivatives such as 11-13 gluco-oligomers, wherein the molecular structures that activate or
   bind to NOD2 include, but are not limited to, peptidoglycans and its derivatives such as muramyl dipeptide and muramyl tripeptide,
   wherein the PET imaging agent is selected from 89Zr, 1241, 64Cu, 18F and 86Y, and wherein the PET imaging agent is complexed with nanobiologic using a suitable chelating agent to form a stable drug-agent chelale.
   wherein said nanobiologic, in an aqueous environment, self-assembles into a nanodisc or nanosphere with size between about 8 nm and 400 nm in diameter,
   wherein the nanoscale assembly delivers the stable drug-agent chelate to myeloid cells, myeloid progenitor cells or hematopoietic stem cells in bone marrow, blood and/or spleen of the patient, and
(ii) performing PET imaging of the patient to visualize biodistribution of the stable drug-agent chelate within the bone marrow, blood, and/or spleen of the patient’s body.

In a non-limiting preferred embodiment, the method of radiopharmaceutical imaging comprises an additional step of administering to said patient a checkpoint inhibitor either concurrently with, or a specified period after the nanobiologic composition,

whereby promoting the hyper-responsive innate immune response caused by trained immunity improves the efficacy of checkpoint inhibitor therapy.

An exemplified protocol using 89Zr is set forth in Example 5.

Further, ex vivo methods may be used to quantify tissue uptake of the 89Zr labeled nanoparticles using gamma counting or autoradiography to validate the imaging results.

This also provides a novel approach to autoradiography-based histology, which allows the evaluation of the nanomaterial’s regional distribution within the tissue of interest by comparing the radioactivity deposition pattern -obtained by autoradiography- with histological and/or immunohistochemical stems on the same or adjacent sections.

Currently, the most commonly used methods to assess nanotherapeutics ‘In vivo behavior rely on fluorescent dyes However, these techniques are not quantitative due to autofluorescence, quenching, FRET, and the high sensitivity of fluorophores to the environment (e.g., pH or solvent polarity). The integration of magnetic resonance imaging imaging agents as nanoparticle labels has been trialed, but requires high payloadS and dosing, compromising the integrity of nanoparticle formulations. Nuclear imaging agents do not have these shortcomings, with 89Zr being especially suited due to its emission of positrons necessary for PET imaging, as well as its relatively long physical hall-life (78.4 hours), which allows for longtudinal studies of slow-clearing substances and eliminates the need for a nearby cyclotron.

Th approach described herein provides an excellent way to functionalize nanobiologics using 89Zr. DSPE-DFO represents a stable way to anchor the DFO chelator into lipid mono- or bilayers. In addition, as DFO is present on the outside of the nanoparticle platform, the nanoparticles can be labeled after they are formulated. This eliminates the need to perform their formulation under radio- shielded conditions, and reduces the amount of activity that needs to be employed. Fastly, the mild conditions with which DSPE-DFO is incorporated, and 89Zr introduced, are compatible with a wide variety of nanoparticle types and formulation methods. In yet another preferred embodiment of the invention, where further stability is desired in the formulation, the invention a lipophilic DFO derivative, named C34-DFO,6 that can be incorporated following the same protocol.

In yet a further non-limiting preferred embodiment of the invention, the invention includes radiolabeled protein-coated nanoparticles prepared by first formulating the particles, then functionalizing the protein component with commercially available p-NCS-Bz-DFO, and finally introducing 89Zr using our general procedure.

### Trained Immunity

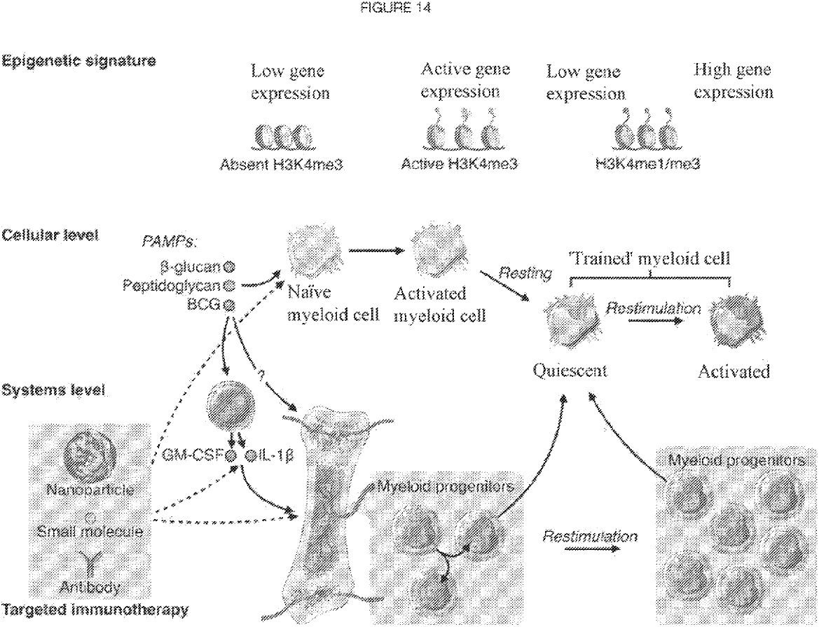

FIG. 14 is an illustration of an up-to-date schematic of processes that control trained immunity, at the epigenetic, cellular and systems level. The originally identifiedtrainers ‘include the fungal PAMP b-glucan and the bacterial PAMP peptidoglycan/BCG. Trained immunity is epigenetically regulated, resulting in a stronger response upon restimulation. Bone marrow progenitors can get stimulated to produce “trained ‘myeloid cells for a prolonged period of time, thereby providing a compelling framework for durable therapeutic interventions.

An in vitro model, in which human monocytes are exposed to either C. albicans or b-glucan, showed genome- wide changes in epigenetic marks, including H3K4met, H3K4me and HSK27Ac (FIG. 14, top). Other studies identified BCG and peptidoglycans as inducers of these trained immunity-associated epigenetic modifications, albeit through the NOD2-dependent pathway. In addition to these epigenetic modifications, cellular metabolism pathways are simultaneously upregulated. In fact, these metabolic changes enhance the cell’s capacity to modulate the function of certain epigenetic enzymes. Upon b-glucan training, a dectinl/Akt/mToR/HIF-la pathway switches cellular metabolism from oxidative phosphorylation to glycolysis, which is associated with a reduced basal respiration rate, increased glucose consumption and higher lactate production.

Although these epigenetic and metabolic changes nicely describe an individual myeloid cell’s increased response to a secondary insult, how this innate immune memory was preserved over a prolonged period of time remained unclear until quite recently. Monocytes have a lifespan of only a few days, whereas trained immunity’s protective function is preserved for much longer, up to several months or almost a year in patients. The most recent insights unveil that on a systems level, trained immunity is a functional program that is also induced in specific hematopoietic stem and progenitor cells (FIG. 14, bottom). Upon administering b-glucan in mice, more myeloid-biased multipotent progenitors (MPPs) and long-term hematopoietic stem cells (LT-FISCs) in the bone marrow may be observed. Various cell proliferation-associated pathways, including cell cycle genes, cholesterol biosynthesis and glycolysis, were upregulated, and these increases were identified as IT-lb- and granulocyte/macrophage colony-stimulating factor (GM-CSF)-dependent. The longevity of these effects was found to persist for up to a month, while transplanting hematopoietic stem cells from b-glucan-trained mice introduced myelopolesis in untrained recipients. Similar observations have been made after administering BCG.

Because trained immunity is a property of myeloid-biased progenitor cells, nanomaterials that are designed to accumulate in bone marrow progenitors for inducing long-term therapeutic effects targeting trained immunity are illustrated.

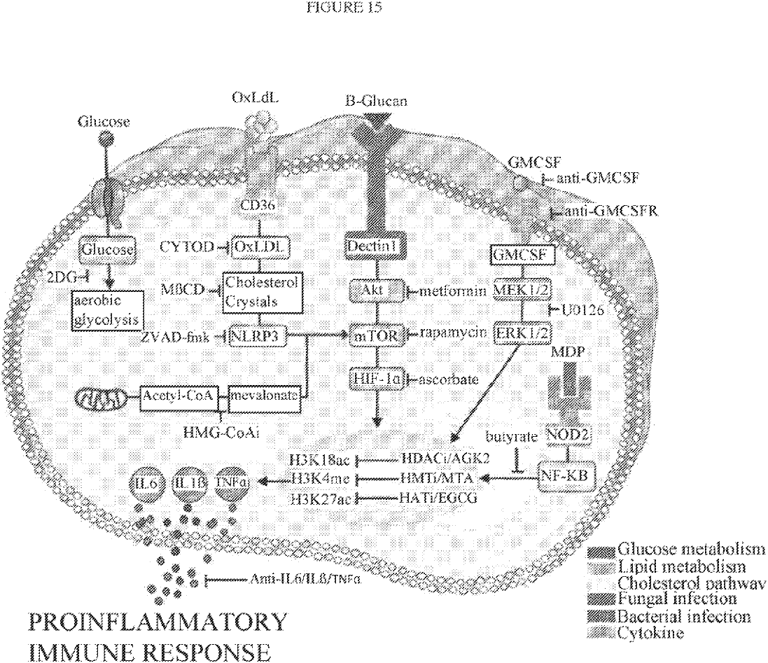

FIG. 15 is an illustration of a cell showing trained immunity is regulated at the cellular level by bacterial, fungal and metabolic pathways, resulting in epigenetic modifications that underlie cytokine secretion.

Immunological signaling events leading to trained immunity phenotype

The induction of trained immunity by microbial ligands is facilitated by specific receptor signaling pathways, that subsequently activate metabolic, epigenetic and transcriptional events. An overview of the most important pathways currently identified is presented in Figure.

### DectinDependent Fungal Pathway

Innate immune cells elicit non-specific immune responses to exogenous pathogens after recognizing b-Glucans. Present in the fungal cell wall, b-Glucans are glucose polymers that are recognized by macrophages as PAMPs through the C-type lectin receptor Dectin-I5I. Macrophage activation via Dectin-1 induces specific epigenetic marks that leads to trained immunity (FIG. 15, red pathway). This activation pathway is typical for fungal infections that can be exploited for therapeutic interventions; non- lethal infection with C. albicans is an example. As mentioned in the introduction, C. albicans has been shown to protect mice against lethal candidiasis through monocyte-dependent trained immunity.

### NOD2-Dependent Bacterial Pathway

Peptidoglycan is a PAMP that synergizes with endotoxin to cause inflammatory cytokine release. The peptidoglycan minimal bioactive motif common to all bacteria is muramyl dipeptide (MDP). Innate Immune cell activation by MDP the cytoplasmic PRR nuclectide binding oligomerization domain 2 (NOD2) to engage. NOD2 activation and signaling through NF-Kb stimulates epigenetic rewiring of macrophages and induces trained immunity 19 (FIG. 15, green pathway). This trained immunity activation pathway is characteristic of bacterial infections, such as the BCG vaccine, which results in proinflammatory cytokine production. The non-specific protective effects of BCG are exploited as immunotherapy for non-invasive bladder cancer.

### Oxidized Low-Density Lipoprotein

Lipid metabolism may also lead to the induction of trained immunity. Oxidized low-density lipoprotein (oxLDL) is a DAMP that binds to the cell surface receptor CD36. Once internalized and released into the cytoplasm, oxLDL may lead to the formation of cholesterol crystals, which activate the NLRP3 inflammasome. A recent report highlighted the critical role of NRLPS activation because of the consumption of a western diet by Ldlr -/- mice, establishing a mechanistic link between oxLDL-induced trained immunity and cardiovascular diseases through the activation of the inflammasome. While oxLDL induces long-lasting proinflammatory phenotype in monocytes and accelerates atherosclerosis, the histone methyltransferase inhibitor methylthloadenosine completely abolishes the training induced by oxLDL.

### Metabolic and Epigenetic Rewiring During Induction of Trained Immunity

Among trained immunity’s effects, one of the most important processes is rewiring innate immune cells ‘metabolism. A key part to this rewiring is the metabolic switch from oxidative phosphorylation towards aerobic glycolysis, which results in Innate immune cell activation and pro-inflammatory cytokine secretion. Candida albicans and b-glucan induce this specific metabolic process through a AKT/mTOR/Hif-la pathway. In addition, BCG vaccination induces immunometabolic activation and epigenetic remodeling, with inhibition of glycolysis by 2-deoxyglucose (2-DG) during BCG training abrogating the increased cytokine production (FIG. 15, purple pathway). The pharmacological modulation of rate-limiting glycolysis enzymes impedes the histone marks HSK4me3 and HSK9me3 underlying both b-glucan and BCG-induced trained immunity.

Another important metabolic event in trained monocytes is the anabolic repurposing of the Krebs cycle towards synthesizing cholesterol and phospholipids from citrate and acetyl CoA. The cholesterol synthesis pathway is upregulated after b-glucan training, with restricted cholesterol synthesis by fluvastatin downregulating HSK4me3 and preventing pro-inflammatory cytokine production and trained immunity. Synthesizing the cholesterol metabolite mevalonate is very important in this process, as trained immunity is prevented enzyme inhibitors downstream of 3-hydroxy-3-methyl-glutaryl-coenzyme A (HMG- CoA)-reductase61 (FIG. 15, yellow pathway). Inhibiting glycolysis with 2-DG, the mTOR pathway with rapamycin, and histone methylation with methyltioadenosine (MTA, a methyltransferase inhibitor) prevent mevalonate-induced trained immunity, indicating a delicate balance between molecular, metabolic and epigenetic control of trained macrophages.

The Krebs cycle is replenished by glutaminolysis. Interestingly, this leads to accumulated succinate and especially lumarate, which are co-factors for important epigenetic enzyme families. In this respect, succinate curbs JMJD3, leading to enhanced H3K27 trimethylation of particular genes (e.g., those associated with the M2 phenotype). However, JMJD enzyme expression did not differ in trained monocytes. In contrast, fumarate inhibits KDM5 histone demethylases: both the expression and function of KDMS have been shown to be blocked/stymied in trained monocytes60. Because KDMS is a demethylase of H3K4 methylation, its suppression permits long-term stability of this important mark of open chromatin and thus facilitates gene transcription.

### Promoting Trained Immunity

BCG induced trained immunity through a NOD2-dependent bacterial pathway. NOD2 is an intracellular PRR that is activated by peptidoglycans, which are polymeric structures of sugars and amino acids that are integral to the bacterial cell wall. The smallest molecular structure capable of inducing a NOD2- dependent immune response is muramyl dipeptide (MDP). MDP is a synthetic peptide conjugate comprised of N-acetyl muramic acid and the short amino acid chain of L-alanine D-isoglutamine dipeptide.

Alternatively, trained immunty can be induced by fungal pathogens through the dectin-l pathway. Dectin-1 is a C-type lectin transmembrane signaling receptor that can be activated by polysaccharides rich in b1,3- or both b1,3- and b1,6-11101ed glucose, known as b-glucans. Other dectin-I-activating polysaccharides, including a liposomal formulation, were extensively studied by Palma and colleagues, who found that I,3-linked glucose oligomers, with a minimum length of 10- or 1 I-mers, were required for dectin-I binding. Consequently, and unlike NOD2 binding, a small molecule ligand is not available for dectin-I-dependent trained immunity induction.

In addition to PAMP-related mechanisms, metabolic′trainers’, such as uric acid and oxLDL, have been shown to induce trained immunity through mTOR signaling and phosphorylation of protein kinase B (AKT). This implies that uric acid itself can be used to induce trained immunity. Although the exact mechanism by which oxLDL induces training remains a topic of investigation, Christ and colleagues acquired compelling evidence for the importance of the NLRP3 inflammasome and the downstream IL-1R signaling pathway, thereby underlining IL-1 bA critical role. Also interesting in the context of oxLDL is the recently discovered role of the cholesterol synthesis intermediate mevalonate. Bekkering and colleagues found that mevalonate induces training via activation of the IGF-1 receptor (IGF-1R) and mTOR and subsequent histone modifications. Mevalonic acid, additionally augmented by 6-fluoromovalonate, may therefore be pharmacologically employed to induce trained immunity. As research continues, currently unknown pathways and molecular structures - including other bacterial and fungal derivatives as well as viral PAMPs - that promote trained immunity will likely be identified.

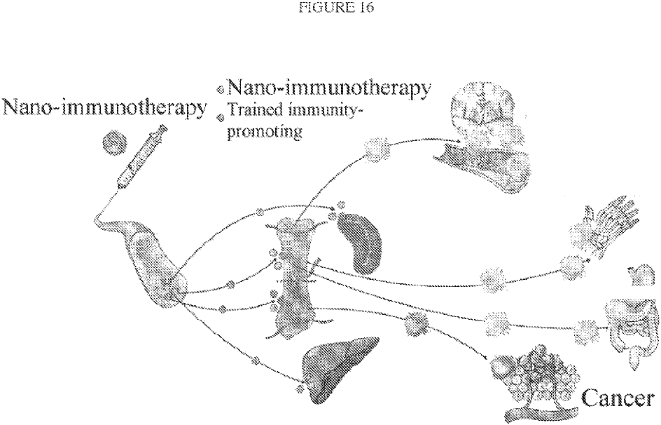

FIG. 16 is an illustration of an overview of processes and show bone marrow-avid nanomaterials that either inhibit (green) or promote (red) trained immunity can be employed to prime the immune system and treat a variety of conditions, ranging from cardiovascular disease and its clinical consequences, autoimmune disorders, to sepsis and infections, as well as cancer.

Nanoparticle delivery vehicles can enhance the percentage of a drug reaching its intended target and improve a therapeutic agent’s toxicity profile. Moreover, the nanoparticle delivery vehicle may facilitate drugs ‘cellular internalization, which is particularly relevant for nucleotide therapy. Moreover, nanoparticles can protect drugs from being prematurely metabolized or degraded.

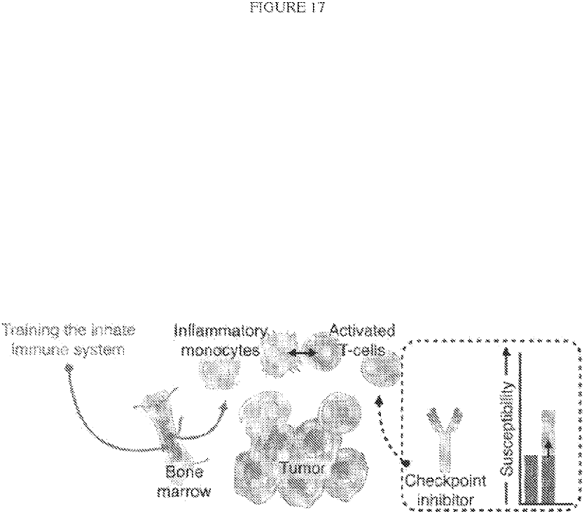

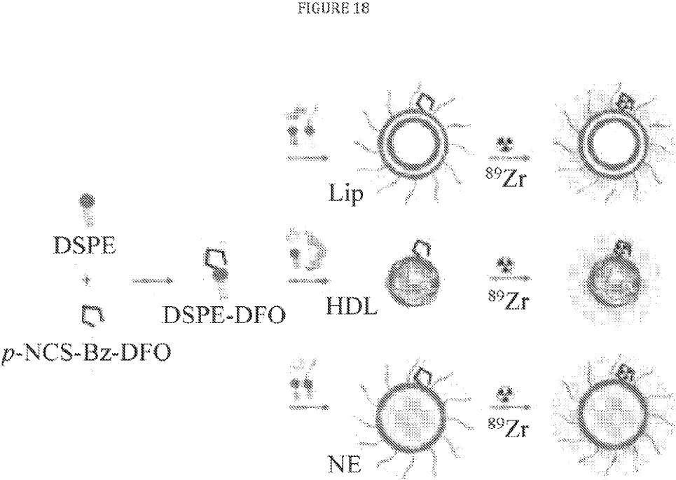

FIG. 17 is an illustration of priming the immune system’s susceptibility toward immune checkpoint blockade therapy can be achieved by promoting trained immunity.

For example, it is increasingly evident that for a certain tumor type, checkpoint blockade immunotherapy only benefits a subset of patients. The pooled analysis of the KEYNOTE-001127 trial found that approximately 34% of late stage melanoma patients had an objective response, while 6% of the patients were full responders. Additionally, in a variety of other malignancies, including prostate and ovarian cancer, checkpoint- inhibitor drugs exert very little therapeutic benefit.

Recent work on peripheral blood from patients has uncovered- using high-dimensional single-cell mass cytometry and a bioinformatics pipeline- that the frequency of classically activated monocytes predicts therapeutic response. Yet high levels of immunosuppressive myeloid cells lead to T-cell dysfunction and failure to respond to checkpoint blockade immunotherapy. We foresee that trained immunity-promoting therapies can promote systemic and tumor-accumulated classically activated monocytes, thereby enhancing susceptibility to checkpoint-inhibitor drugs, as outlined in FIG. 17.

### EXAMPLES

The following examples are included to demonstrate embodiments of the disclosure. The following examples are presented only by way of illustration and to assist one of ordinary skill in using the disclosure. The examples are not intended in any way to otherwise limit the scope of the disclosure. Those of ordinary skill in the art should, in light of the present disclosure, appreciate that many changes can be made in the specific embodiments which are disclosed and still obtain a like or similar result without departing from the spirit and scope of the disclosure.

### Example 1 - Microfluidizer Assembly 1

This example demonstrates the preparation of a pharmaceutical composition comprising STIMULATOR and the nanoscale assembly in which the STIMULATOR concentration is 4-8 mg/mL in the nanoscale assembly/emulsion and the formulation is made on a 300 mL scale. STIMULATOR (2400 mg) is dissolved in 12 mL of chloroform/t-butanol. The solution is then added into 288 mL of a nanoscale assembly solution (3% w/v) including a mixture of POPC/PHPC phospholipids, apoA-I, tricaprylin, and cholesterol. The mixture is homogenized for 5 minutes at 10,000-15,000 rpm (Vitris homogenizer model Tempest I.Q.) in order to form a crude emulsion, and then transferred into a high pressure homogenizer. The emulsification is performed at 20,000 psi while recycling the emulsion. The resulting system is transferred into a Rotavap, and the solvent is rapidly removed at 40° C. at reduced pressure (25 mm of Hg). The resulting dispersion is translucent. The dispersion is serially filtered through mutiple filters. The size of the filtered formulation is 8-400 nm.

### Example 2- Microfluidizer Assembly 2

This example demonstrates the preparation of a pharmaceutical composition comprising STIMULATOR and the nanoscale assembly in which the STIMULATOR concentration is 4-8 mg/mL in the nanoscale assembly/emulsion and the formulation is made on a 300 mL scale. STIMULATOR (2400 mg) is dissolved in 12 mL of chloroform/t-butanol. The solution is then added into 288 mL of a nanoscale assembly solution (3% w/v) including a mixture of POPC/PHPC phospholipids, a peptide mimetic of apoA-I, a mixture of C16-C20 triglycerides, a mixture of cholesterol and one or more sterol esters, and a hydrophobic polymer. The mixture is homogenized for 5 minutes at 10,000-15,000 rpm (Vitris homogenizer model Tempest I.Q.) in order to form a crude emulsion, and then transferred into a high pressure homogenizer. The emulsification is performed at 20,000 psi while recycling the emulsion.

The resulting system is transferred into a Rotavap, and the solvent is rapidly removed at 40° C. at reduced pressure (25 mm of Hg). The resulting dispersion is translucent. The dispersion is serially filtered through multiple filters. The size of the filtered formulation is 35-100 nm.

### Example 3 - Lyophilization of Nanosiologics of Examples 1 and 2

The nanobiologic is formed as in either of the above examples. The dispersion is further lyophilized (FTS Systems, Dura-Dry mR, Stone Ridge, N.Y.) for 60 hours. The resulting lyophilization cake is easily reconstitutable to the original dispersion by the addition of sterile water or 0.9% (w/v) sterile saline. The particle size after reconstitution is the same as before lyophilization.

### Example 4- Nanobiologic Treatment Either Alone or in Combination With Checkpoint Inhibitors Was Effective in Reducing Tumor Size and Increasing Trained Immunity

MTP-HDL nanobiologics were formulated from the phospholipid DMPC. cholesterol and muramyl tripeptide phosphatidylethanolamine (MTP-DSPE) as described herein.

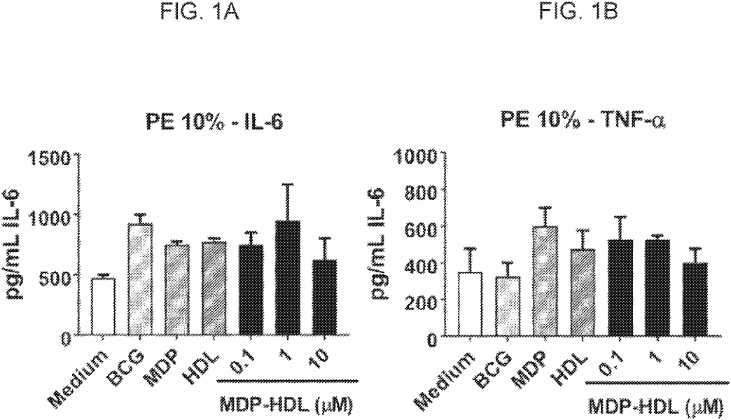

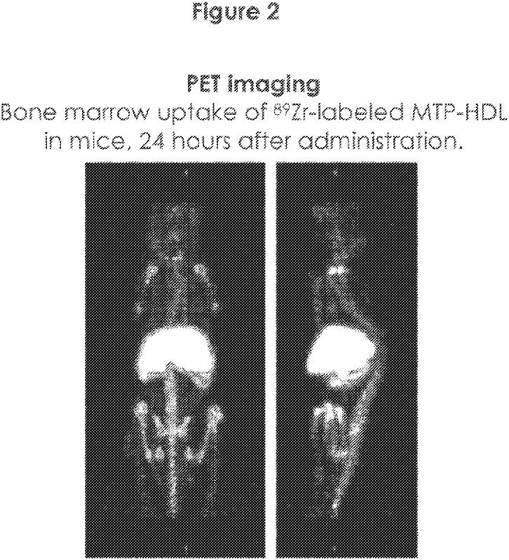

In an in vitro assay, in which monocytes were exposed for 24 hours to the respective ‘training ’ agents (Beta-glucan, MDP or MTP-HDL) followed by resting and restimulation with LPS, it was shown that MTP-HDL induces trained immunity in human monocytes in vitro (FIG. 1), as was appreciated from increased IL-6 and TNF-a secretion. In vivo PET-CT was employed to quantitatively and noninvasively study the in vivo behavior of 89Zr-labeled MTP-HDL nanobiologics. A high avidity towards the bone marrow (FIG. 2) and MTP-HDL ‘s presence in hematopoietic stem cells and myeloid progenitors was observed.

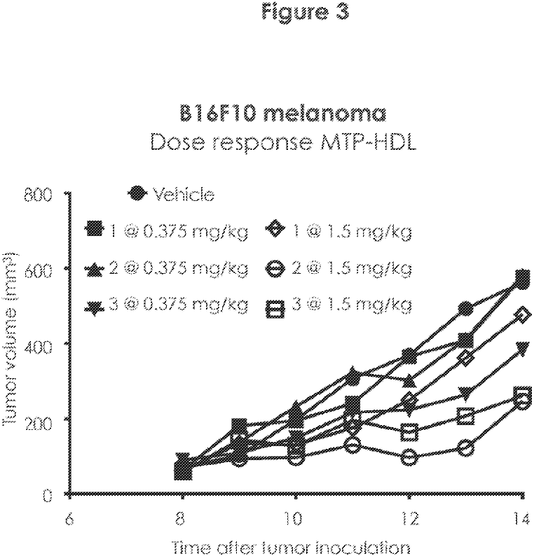

A dose response study, involving different regimens, i.e., 1, 2 or 3 injections in low (0.375 mg/kg MTP) and high dose (1.5 mg/kg MTP) and a non-functionalized HDL control group, was executed in C57BL/6 bearing B16F10 melanoma tumors. A dose- and regimen-dependency was observed, in the absence of any adverse effects (FIG. 3). As shown in FIG. 3, all doses of the MTP-HDL reduced tumor volume with the higher dosages of 1.5 mg/kg administered 2 or 3 times reducing the tumor volume the most effectively.

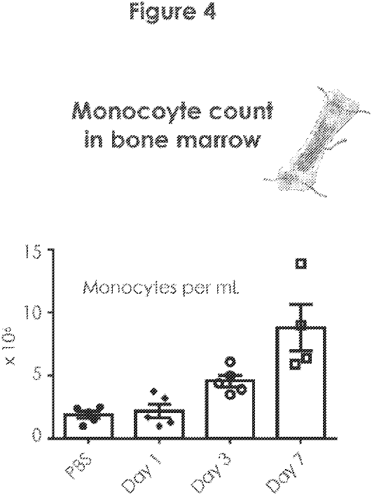

The most effective regimen, consisting of 3 intravenous MTP-HDL injections at 1.5 mg/kg (MTP), was applied to regular C57BL/6 mice. At several time points after the last MTP-HDL injection, mice were sacrificed and the number of monocytes was quantified. A clear increase in monocyte numbers as a result of MTP-HDL treatment was observed (FIG. 4).

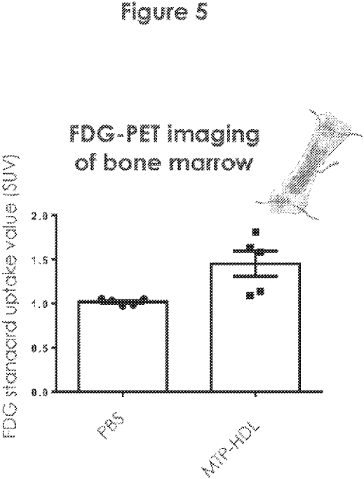

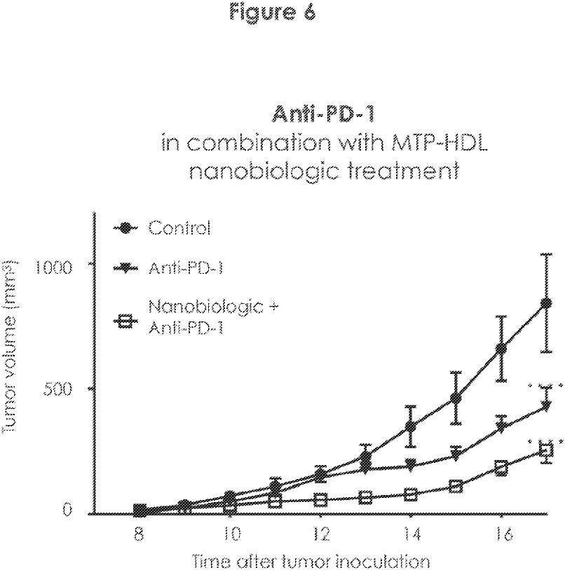

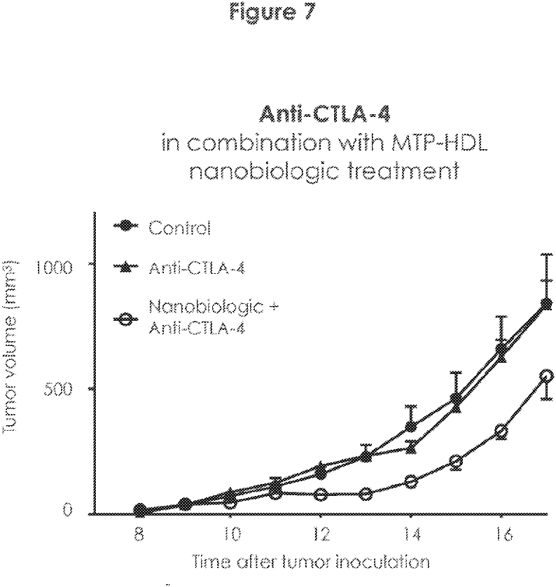

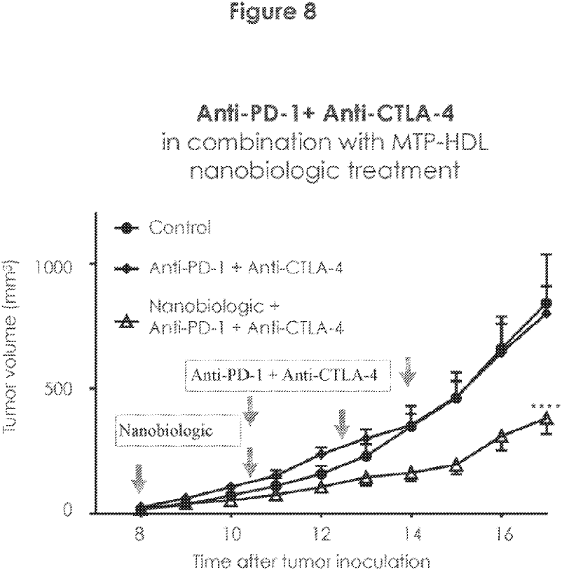

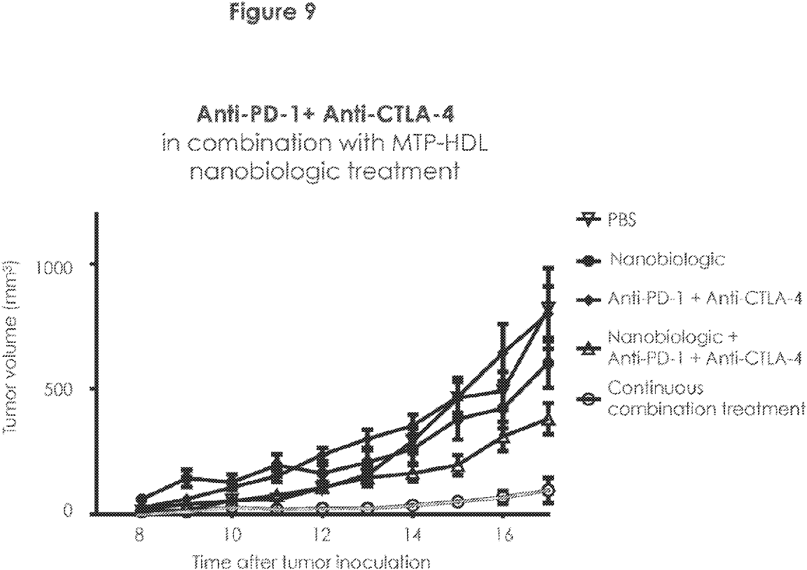

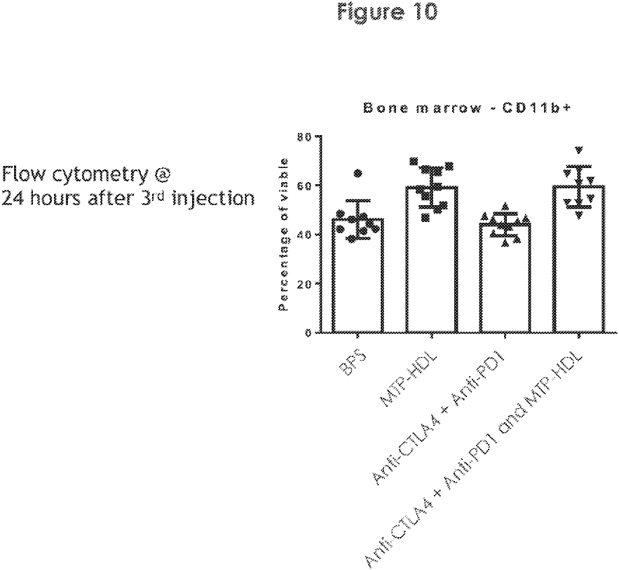

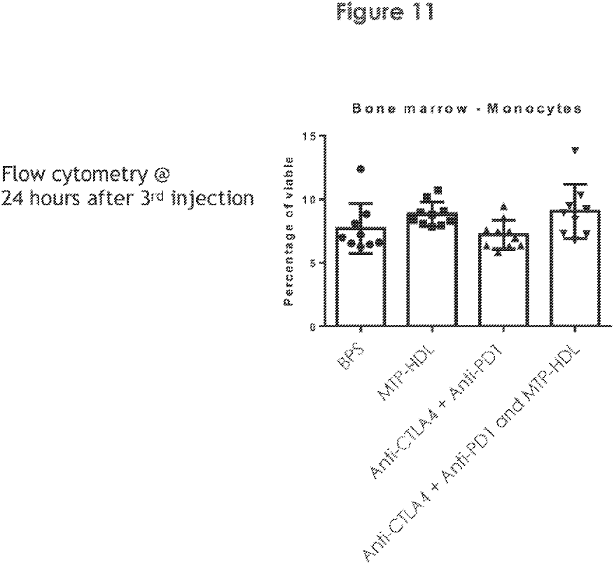

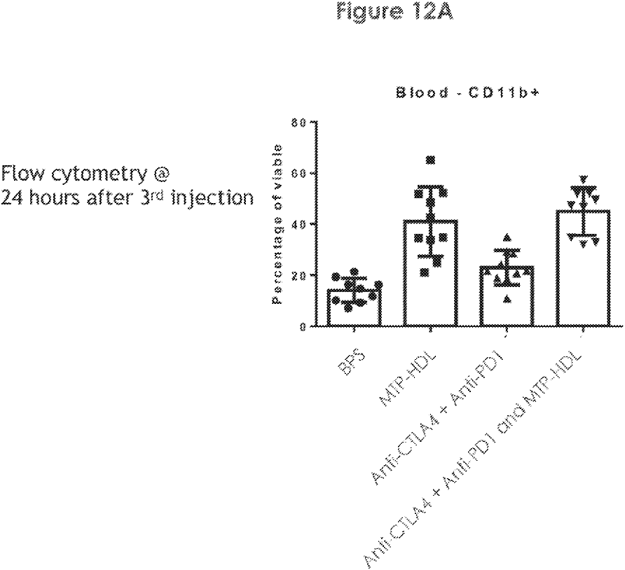

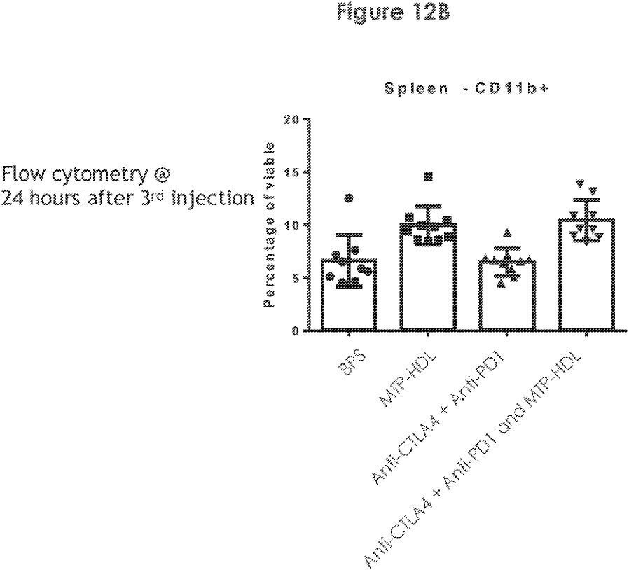

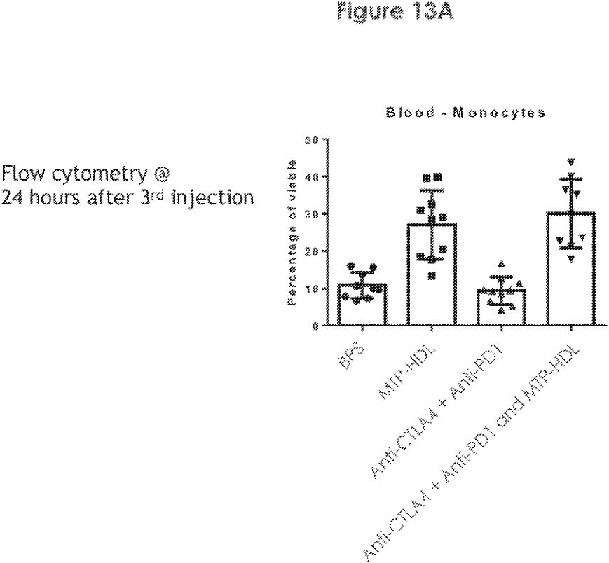

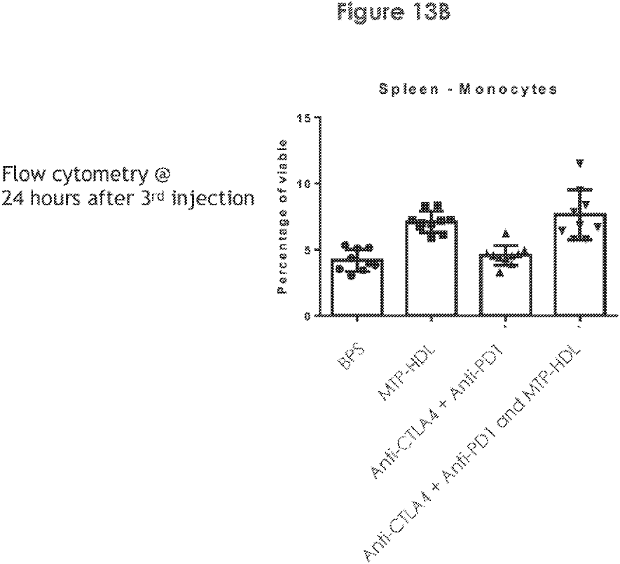

In a separate set of experiments, regular C57BL/6 mice received 3 intravenous MTP-HDL injections at 1.5 mg/kg (MTP), after which they were subjected to FDG-PET imaging of the bone marrow. As FDG is a sugar analog, its uptake is proportionate to metabolic activity, which was found to be higher in bone marrow of mice treated with MTP-HDL (FIG. 5) Therapeutic in vivo studies with MTP-HDL in combination with different checkpoint blockade immunotherapies were conducted. Treatment groups consisted of anti-CTLA-4 (FIG. 6), anti-PD-l (FIG. 7) or the combination of both (FIGS. 8 and 9) at a checkpoint inhibitor drug dose of 200pg with or without the concurrent induction of trained immunity by MTP-HDL The combination of checkpoint inhibition and the MTP-HDL-induced trained immunity resulted in significantly enhanced anti-tumor activity as compared to several controls. Flow cytometry analysis of cells in the blood, bone marrow and spleen not only showed that MDP-HDL alone increased both monocytes and CD1 lb+ cells in all tissues over control and combination anti-CTLA4 and anti- PD 1 therapy but the combination of all three was the most effective in increasing both types of cells in all tissues. See FIGS. 10-13.

### Example 5 - Radiopharmaceutical Labeling of Trained Immunity Promoter Drugs

In a non-limiting example, radiopharmaceutical labeling of trained immunity promoter drugs/molecules can be achieved through various types of chelators, primarily deferroxamine B (DFO) which can form a stable chelate with 89Zr through the 3 hydroxamate groups. Generally, phospholipids are conjugated with a chelator compound, the nanobiologic is prepared with the promoter drug or molecule, and finally, the radioisotope is complexed with the nanobiologic (that already has the chelator attached).

This protocol includes the synthesis of DSPE-DFO, obtained through reaction of the phospholipid DSPE and an isothiocyanate derivative of the chelator DFO (p-NCS-Bz-DFO), its formulation into nanobiologics, and nanoemulsions, and the subsequent radiolabeling of these nanoformulations with 89Zr.

The radioisotope 89Zr was chosen due to its 3.3-day physical decay half-life, which eliminates the need for a nearby cyclotron and allows studying agents that slowly clear from the body, such as antibodies. Although both are contemplated as workable herein, 89Zr’s relatively low positron energy allows a higher imaging resolution compared to other isotopes, such as 124l.

The 89Zr labeling of the nanotherapeutics enables non-invasive study of in vivo behavior by positron emission tomography (PET) imaging in patients.

The protocol includes the following steps:
Conjugation of the chelator deferoxamine B (DFO) to the phospholipid DSPE, to thereby form a lipophilic chelator (DSPE-DFO) that readily integrates in different lipid nanoparticle platforms (~0.5 wt%);
Preparation of nanoscale assembly formulations (using sonication, nanoemulsions using hot dripping, or using microfluidics) that have DSPE-DFO incorporated; and
Labeling of DSPE-DFO containing lipid nanoparticles with 89Zr, performed by mixing the nanoparticles for 30-60 minutes with 89Zr-oxalate at pH~7 and 30-40° C. in PBS.

Additionally, purification and characterization methods are be used to obtain radiochemically pure 89Zr-labeled lipid nanoparticles. Purification is typically be performed using either centrifugal filtration or a PD- 10 desalting column, and subsequently assessed using size exclusion radio-HPLC. Typically, the radiochemical yield is >80%, and radiochemical purities >95% are normally obtained.

General imaging strategies are used to study 89Zr-labeled nanobiologic in vivo behavior by PET/CT or PET/MRI.

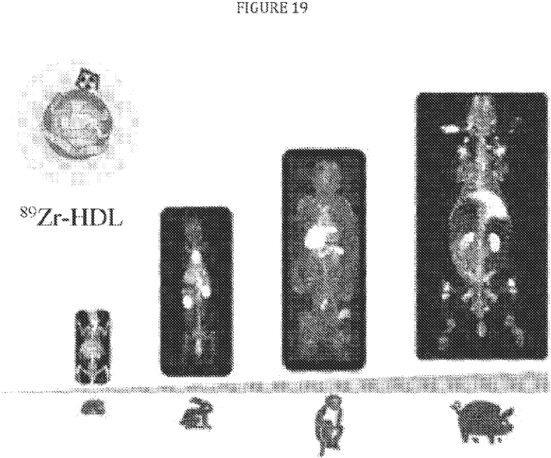

FIG. 19 shows PET imaging using a radioisotope delivered by nanobiologic and shows accumulation of the nanobiologic in the bone marrow and spleen of a mouse, rabbit, monkey, and pig model.

### Example 6- Synthesis of Nanobiologics Including Prodrugs

### Materials and Methods

All chemicals were purchased from Sigma Aldrich, Medchem Express or Selleckchem, PES syringe filters were obtained from Celltreat. A NE-1002X model microfluidic pump from World precision instruments was used in combination with Zeonor herringbone mixers from Microfluidic-chipshop (#14-1038-0187-06). Particles were purified using a 100 kDa MWCO 20 mL Vivaspin centrifugal filter. Dialysis bags were from Thermo Scientific. The ApoA-I protein was puritied in house using a previously published procedure. Spectroscopic quantification of ApoA-I was performed on a BioTek Cytation 3 imaging plate reader using the Bradfort assay. DLS and Zeta potential measurements were performed on a Brookhaven instrument corporation ZetaPals analyzer, the mean of the number distribution was taken to determine particles sizes. 1 H and 13C NMR samples were analyzed using a Bruker 600 ultreshieid magnet connected to a Bruker advance 600 console, data was processed using Topspin version 3.5 pl 7. Quantitative analysis of all drugs was performed by HPLC analysis using a Shimadzu UFLC apparatus equipped with either a Cis or CN column. Acetonitrile and water were used as mobile phase and compounds were detected with an SPD-M20a diode array detector.

### Synthesis of the ~ 35 Nm Nanobiologies

From 10 mg/ml stock solutions in chloroform, I-palmiloyl-2-oleoyl-sn-glycero-3-phosphocholine (POPC, 250 pL), I-palmitoyl-2-hydroxy-en-glycero-3-phosphocholine (PHPC, 65 pL), cholesterol (15 pL), tncaprylin (1000 pL) and drug or (pro-)drug (65 pL), were combined in a 20 ml vial and dried under vacuum. The resulting film was redissolved in a acetonitrile methanol mixture (95 % : 5 %, 3 mL total volume). Separately, a solution of ApoA-I protein in PBS (0.1 mg/ml) was prepared. Using a microfluidic set-up, both solutions were simultaneously injected into a herringbone mixer, with a flow rate of 0.75 ml /min for the lipid solution and a rate of 6 ml /min for the ApoA-I solution. The obtained solution was concentrated by centrifugal filtration using a 100 MWCO Vivaspin tube at 4000 rpm to obtain a volume of 5 mL. PBS (5 mL) was added and the solution was concentrated to 5 mL, again PBS (5 mL) was added and the solution was concentrated to approximately 3 mL. The remaining solution was filtered through a 0.22 p PES syringe filter to obtain the final nanobiologic solution. To obtain nanobiologies for FACS measurements, 3,3′-Dioactadecyloxacarbocyanine perchlorate (DIO-Cis, 0.25 mg) was added to the acetonitrile solution. To obtain nanobiologics for 89Zr labeling, DSPE-DFO (50 pg) was added to the acetonitrile solution (made in house). To scale up the nanobiologic synthesis the above procedure was simply repeated until sufficient amounts were produced.

### Synthesis of the Nanobiologics (« 15 Nm)

For the synthesis of the 15 nm sized nanoparticles a similar microfluidic procedure as for the 35 nm sized particles was used. Here, the acetonitrile mixture contained (again from 10 mg/ml stock solutions): POPC (260 pL), PHPC (15 pL), Cholesterol (13 pL) and drug or (pro-)drug (65 pL). The acetonitrile solution was injected with a rate of 0.75 mL/min. The ApoA-I solution (0.1 mg/mL in PBS) was injected with 3 mL/min. To obtain nanobiologics for FACS measurements, DIO-Cis (0.25 mg) was added to the acetonitrile solution. To obtain nanotnologics for 89Zr labeling, DSPE-DFO (50 pg) was added to the acetonitrile solution.

### Synthesis of the Nanobiologics (« 65 Nm)

For the synthesis of the 66 nm sized nanoparticles a similar microfluidic procedure as for the 35 nm sized particles was used. Here, the acetonitrile mixture contained (again from 10 mg/ml stock solutions): POPC (250 pI), Cholesterol (12 pL), Tricaprylin (1400 pL) and drug or (pro-)drug (65 pL). The acetonitrile solution was injected with a rate of 0.75 mL/min. The ApoA-I solution (0.1 mg/ml in PBS) was injected with 4 mL/min. To obtain nanobiologics for FACS measurements, DIO-Cis (0.25 mg) of was added to the acetonitrile solution. To obtain nanobiologics for 89Zr labeling, DSPE-DFO (50 pg) was added to the acetonitrile solution.

### Synthesis of the Nanobiologics (« 120 Nm)

For the synthesis of the 120 nm sized nanoparticles a similar microfluidic procedure as for the 35 nm sized particles was used. Here, the acetonitrile mixture contained (again from 10 mg/ml stock solutions): POPC (100 mt), Cholesterol (10 pL). Tricaprylin (4000 pL) and drug or (pro-)drug (65 pL). The acetonitrile solution was injected with a rate of 0.75 mL/min. The ApoA-I solution (0.1 mg/ml in PBS) was injected with 1.5 mL/min. To obtain nanobiologics for FACS measurements, DIO-Cix (0.26 mg) of was added to the acetonitrile solution. To obtain nanobiologics for 89Zr labeling. DSPE-DFO (50 pg) was added to the acetonitrile solution.

### Size Stability of the Four Different Sizes of Nanoparticles

An aliquot (10 pL) of the final particle solution was dissolved in PBS (1 mL), filtered through a 0.22 pm PES syringe filter and analyzed by DLS to determine the mean of the number average size distribution. Samples were analyzed directly upon synthesis of the particles as well as 2, 4, 6, 8, 10 days afterwards.

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Rather, these embodiments are provided so that this disclosure will be thorough and complete, and will fully convey the scope of the invention to those skilled in the art. Like numbers refer to like elements throughout. As used herein the term “and/or” includes any and all combinations of one or more of the associated listed items.

The terminology used herein is for the purpose of describing particular embodiments only and is not intended to limit the full scope of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless defined otherwise, all technical and scientific terms used herein have the same meanings as commonly understood by one of ordinary skill in the art. Nothing in this disclosure is to be construed as an admission that the embodiments described In this disclosure are not entitled to antedate such disclosure by virtue of prior invention. As used in this document, the term “comprising” means “including, but not limited to.”

Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “Open” terms (e.g., the term “including” should be interpreted as “including but not limited to,” the term “having” should be Interpreted as “having at least,” the term “includes” should be interpreted as “includes but is not limited to,” etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal subparts. As will be understood by one skilled in the art, a range includes each individual member.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

Having described embodiments for the invention herein, it is noted that modifications and variations can be made by persons skilled in the art in light of the above teachings. It is therefore to be understood that changes may be made in the particular embodiments of the invention disclosed which are within the scope and spirit of the invention as defined by the appended claims, Having thus described the invention with the details and particularity required by the patent laws, what is claimed and desired protected by Letters Patent is set forth in the appended claims,

1. A method of treating cancer in a patient by inducing trained immunity comprising:
administering to said patient a nanobiologic composition in an amount effective to promote a hyper-responsive innate immune response,
wherein the nanobiologic composition comprises a nanoscale assembly, wherein the nanoscale assembly is a multi-component carrier composition comprising: (a) a phospholipid, and (b) an apolipoprotein A-1 (apoA-1) or a peptide mimetic of apoA-1 and (c), a NOD2 activator
wherein said nanoscale assembly is a nanodisc or nanosphere with size between about 8 nm and 400 nm in diameter, and wherein the NOD2 activator binds to a NOD2 receptor on one or more of is a myeloid cell and a myeloid cell progenitor cell located in the bone marrow of the patient, .

2. A method of treating a patient by improving the efficacy of a checkpoint inhibitor treatment by inducing trained immunity:
(1) administering to said patient a nanobiologic composition in an amount effective to promote a hyper-responsive innate immune response, wherein the nanobiologic composition comprises (i) a nanoscale assembly, having (ii) an innate immune response promoter drug incorporated in the nanoscale assembly, wherein the nanoscale assembly is a multi-component carrier composition comprising: (a) phospholipids, and, (b) apolipoprotein A-1 (apoA-1) or a peptide mimetic of apoA-1, wherein said nanobiologic, in an aqueous environment, is a nanodisc or nanosphere with size between about 8 nm and 400 nm in diameter, wherein the nanobiologic is functionalized with a molecular structure that activates or binds to the pathogen recognizing receptors Dectin-1 or NOD2 to induce trained immunity in myeloid cells and their stem cells and progenitors in the bone marrow, blood and spleen, wherein the molecular structures that activate or bind to Dectin-1 are chosen from the group consisting of b-glucans, and b-glucan derivatives and wherein the molecular structures that activate or bind to NOD2 are chosen from the group consisting of peptidoglycans and peptidoglycan derivatives, wherein the nanoscale assembly delivers the trained immunity-promoter molecular structures to myeloid cells, myeloid progenitor cells or hematopoietic stem cells in bone marrow, blood and/or spleen of the patient; whereby in the patient a hyper-responsive innate immune response caused by trained immunity is promoted; and
(2) administering to said patient a checkpoint inhibitor;
whereby promoting the hyper-responsive innate immune response caused by trained immunity improves the efficacy of checkpoint inhibitor therapy.

3. (canceled)

4. A method of treating a patient affected by defective trained immunity to promote in said patient a long-term hyper-responsive innate immune response, comprising:
(1) administering to said patient a nanobiologic composition in an amount effective to promote a hyper-responsive innate immune response,
wherein the nanobiologic composition comprises (i) a nanoscale assembly, having (ii) an promoter drug incorporated in the nanoscale assembly,
wherein the nanoscale assembly is a multi-component carrier composition comprising:
(a) phospholipids, and, (b) apoA-1 or a peptide mimetic of apoA-1,
wherein the promoter drug is a molecular structure that activates or binds to the pathogen recognizing receptors Dectin-1 or NOD2 to induce trained immunity in myeloid cells and their stem cells and progenitors in the bone marrow, blood and spleen, wherein the molecular structures that activate or bind to Dectin-1 are chosen from the group consisting of b-glucans, and b-glucan derivatives and wherein the molecular structures that activate or bind to NOD2 are chosen from the group consisting of peptidoglycans and peptidoglycan derivatives, wherein said nanobiologic, in an aqueous environment, self-assembles into a nanodisc or nanosphere with size between about 8 nm and 400 nm in diameter,
wherein the nanoscale assembly delivers the drug to myeloid cells, myeloid progenitor cells or hematopoietic stem cells in bone marrow, blood and/or spleen of the patient, and whereby in the patient the hyper-responsive innate immune response is promoted, and optionally; (2) administering to said patient a checkpoint inhibitor after administering the nanobiologic composition, whereby promoting the hyper-responsive innate immune response caused by trained immunity improves the efficacy of checkpoint inhibitor therapy.

5. A method of radiopharmaceutical imaging an accumulation of a promoter drug within bone marrow, blood, and/or spleen, of a patient affected by trained immunity, comprising:
(1) administering to said patient a nanobiologic composition in an amount effective to promote a hyper-responsive innate immune response,
wherein the nanobiologic composition comprises (i) a nanoscale assembly, having (ii) an promoter drug incorporated in the nanoscale assembly, and (iii) a positron emission tomography (PET) imaging agent incorporated in the nanoscale assembly,
wherein the nanoscale assembly is a multi-component carrier composition comprising:
(a) phospholipids, and
(b) apoA-1 or a peptide mimetic of apoA-1,
wherein the promoter drug is a molecular structure that activates or binds to the pathogen recognizing receptors Dectin-1 or NOD2 to induce trained immunity in myeloid cells and their stem cells and progenitors in the bone marrow, blood and spleen, wherein the molecular structures that activate or bind to Dectin-1 are chosen from the group consisting of b-glucans, and b-glucan derivatives and wherein the molecular structures that activate or bind to NOD2 are chosen from the group consisting of peptidoglycans and peptidoglycan derivatives, wherein the PET imaging agent is selected from the group consisting of ⁸⁹Zr, ¹²⁴I, ⁶⁴Cu, and ⁸⁶Y, and wherein the PET imaging agent is conjugated to the promoter drug using a suitable chelating agent to form a stable drug- agent chelate, wherein said nanobiologic, in an aqueous environment, self-assembles into a nanodisc or nanosphere with size between about 8 nm and 400 nm in diameter,
wherein the nanoscale assembly delivers the stable drug-agent chelate to myeloid cells, myeloid progenitor cells or hematopoietic stem cells in bone marrow, blood and/or spleen of the patient; and
(2) performing PET imaging of the patient to visualize biodistribution of the stable drug- agent chelate within the bone marrow, blood, and/or spleen of the patient’s body.

6. The method of claim 1, wherein the nanoscale assembly further comprises (d) a hydrophobic matrix comprising one or more triglycerides, fatty acid esters, cholesterol or a combination thereof.

7. (canceled)

8. The method of claim 5, wherein the method of radiopharmaceutical imaging comprises an additional step of administering to said patient a checkpoint inhibitor after administering the nanobiologic composition, whereby promoting the hyper-responsive innate immune response caused by trained immunity improves the efficacy of checkpoint inhibitor therapy.

9. The method of claim 1, wherein the hyper-responsive innate immune response is promoted for at least 7 to 30 days.

10. The method of claim 1, wherein the hyper-responsive innate immune response is promoted for at least 30 to 100 days.

11. The method of claim 1, wherein the hyper-responsive innate immune response is promoted for more than 100 days and up to 3 years.

12. The method of claim 1, wherein the patient has cancer of the bladder, blood vessels, bone, brain, breast, cervix, chest, colon, endometrium, esophagus, eye, head, kidney, liver, lymph nodes, lung, mouth, neck, ovaries, pancreas, prostate, rectum, skin, stomach, testis, throat, thyroid, urothelium, or uterus.

13. The method of claim 1, wherein the nanobiologic composition is administered once and wherein the hyper-responsive innate immune response is promoted for at least 30 days.

14. The method of claim 1, wherein the nanobiologic composition is administered at least once per day in each day of a multiple-dosing regimen, and wherein the hyper-responsive innate immune response is promoted for at least 30 days.

15. The method of claim 1, wherein the NOD2 activator is a bacterial peptidoglycan.

16-32. (canceled)

33. The method of claim 15 wherein the bacterial peptidoglycan is a muramyl dipeptide (MDP) or a muramyl tripeptide (MTP).
